(19) United States
(12) Patent Application Publication
(10) Pub. No.: US 2010/0087453 A1
(43) Pub. Date: Apr. 8, 2010

(54) COMPOUNDS, SCREENS, AND METHODS OF TREATMENT

(75) Inventors: Junying Yuan, Newton, MA (US); Chengye Yuan, Shanghai (CN); Alexei Degterev, Brookline, MA (US)

Correspondence Address: CLARK & ELBING LLP, 101 FEDERAL STREET, BOSTON, MA, 02110, US

(73) Assignees: President and Fellows of Harvard College, Cambridge, MA (US); Shanghai Institute of Organic Chemistry, Shanghai (CN)

(21) Appl. No.: 12/444,822

(22) PCT Filed: Oct. 9, 2007
(86) PCT No.: PCT/US07/21525

§ 371 Date: Dec. 16, 2009

Related U.S. Application Data
(60) Provisional application No. 60/850,539, filed on Oct. 10, 2006.

(51) Int. Cl. A61K 31/519; C07D 239/00; C07D 491/14; A61K 31/505; A61P 25/28

(52) U.S. Cl. 514/260.1; 544/250; 544/278; 514/267

(57) ABSTRACT

The present invention features compounds of Formula (I), pharmaceutical compositions, methods of synthesis, and methods for treating diseases and conditions associated with cellular necrosis. Screening assays for identifying compounds useful for treating these conditions are also described.

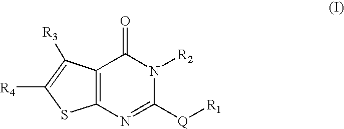

### BACKGROUND OF THE INVENTION

In many diseases, cell death is mediated through apoptotic and/or necrotic pathways. Apoptosis is regulated by an evolutionarily conserved cellular mechanism that proceeds through specific signal transduction pathways common to different cell types. Necrosis, on the other hand, is thought to be an unregulated cellular response to overwhelming stress. Despite the prevalence of necrosis under pathologic conditions, therapeutic strategies to prevent cell death in pathological conditions have targeted apoptosis rather than necrosis, because of the perception that necrosis is an unregulated and nonspecific process and therefore, difficult to be targeted for therapeutic purposes.

While much is known about the mechanisms of action that control apoptosis, control of necrosis is not as well understood. Understanding the mechanisms regulating both necrosis and apoptosis in cells is essential to being able to treat conditions, such as neurodegenerative diseases, stroke, coronary heart disease, kidney disease, and liver disease. A thorough understanding of necrotic and apoptotic cell death pathways is also crucial to treating AIDS and the conditions associated with AIDS, such as retinal necrosis.

Research has shown that caspases play a central role in the induction of apoptosis. Peptide based inhibitors of caspases, such as zVAD-fmk are useful in preventing activation of the apoptotic cell death pathway in cells stimulated to undergo apoptosis by compounds such as TNFα. However, cells treated with zVAD-fmk and these cell death stimuli still die through a caspase-independent form of necrosis.

Although stimulation of the Fas/TNFR death receptor (DR) family triggers a canonical ‘extrinsic’ apoptosis pathway, it was demonstrated that in the absence of intracellular apoptotic signaling, Fas/TNFR is capable of activating a common non-apoptotic death pathway termed “necroptosis” (Vercammen et al., J. Exp. Med. (1998) 188:919-930; Matsumura et al., J. Cell Biol. (2000) 151:1247-1256; Holler et al., J. Nat. Immunol. (2000) 1:489-495; Kawahara, et al. J. Cell. Biol. (1998) 143:1353-1360). Necroptosis is a regulated cell death pathway, activated upon stimulation of FasL/TNFα family of death receptor ligands under the conditions when apoptosis is inhibited, and characterized by morphological features normally attributed to unregulated necrosis. The existence of a regulated cellular necrotic cell death mechanism raises the possibility of specifically targeting the necrotic component of human disease.

The discovery of compounds that prevent caspase-independent cell death would provide useful therapeutic agents for treating conditions in which necrosis occurs, and for preventing the onset of necrosis. These compounds and methods would be particularly useful for treating neurodegenerative diseases, ischemic brain and heart injuries, and head traumas.

### SUMMARY OF THE INVENTION

The present invention features compounds, pharmaceutical compositions, methods of synthesis, and methods for treating a range of conditions, e.g., those in which cell or tissue necrosis is a causative factor or result, those in which loss of proliferative capacity is a causative factor or a result, and those in which cytokines of the TNF-α family are a causative factor or a result.

The invention is directed to a compound of Formula (I):

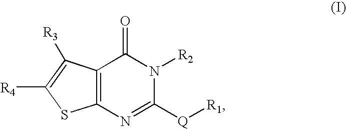

or a pharmaceutically acceptable salt thereof, where

Q is selected from the group consisting of —S—, —S(O)—, and —S(O)₂—;

R₁ is selected from the group consisting of C₁-C₉ alkyl, C₂-C₉ alkenyl, C₂-C₉ alkynyl, C₆-C₁₂ aryl, and C₁-C₁₂ carbonyl;

R₂ is selected from the group consisting of C₁-C₉ alkaryl, and C₆-C₁₂ aryl; and

R₃ and R₄ are, independently, selected from the group consisting of C₁-C₉ alkyl, C₂-C₉ alkenyl, C₂-C₉ alkynyl, C₁-C₉ alkyloxy, and C₁-C₁₂ carbonyl, or R₃ and R₄, combined, form an C₃-C₉ carbocyclic, C₂-C₉ heterocyclic, C₆-C₁₂ aryl, or C₂-C₁₂ heteroaryl, ring system;

with the proviso that compounds where Q is —S—, R₁ is —CH₂CN, R₂ is —C₆H₄(4-OMe), and R₃ and R₄, combined, form an unsubstituted C₆-carbocyclic six-membered ring, are specifically excluded.

The invention is further directed to compounds of Formula (II):

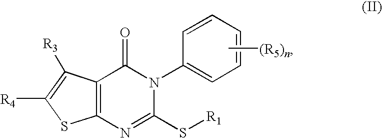

where

R₁, R₃ and R₄ are defined as above;

R₅ is selected from the group consisting of H, C₁-C₉ alkyl, C₂-C₉ alkenyl, C₂-C₉ alkynyl, C₃-C₉ carbocyclyl, C₂-C₉ heterocyclyl, C₂-C₉ heteroaryl, C₆-C₁₂ aryl, C₁-C₉ alkyloxy, C₁-C₉ alkylthio, C₆-C₁₂ arylthio, C₁-C₉ hydroxyalkyl, C₁-C₉ alkyloxy, C₆-C₁₂ aryloxy, C₁₋₁₂ carbonyl, C₁-C₉ fluoroalkyl, C₁-C₉ perfluoroalkyl, halogen, —SH, —OH, —N₃, —NH₂, —NO₂, and CN; and

n is 1, 2, 3, or 4.

The present invention is further directed to compounds of Formula (III):

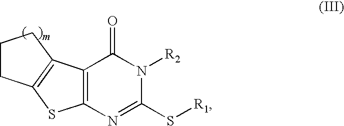

where

R₁ and R₂ are defined as above; and

m is 1, 2 or 3.

The invention is also directed to compounds of Formula (IV):

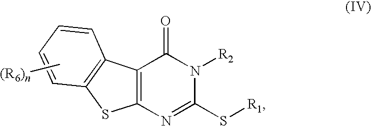

where

R₁ and R₂ are as defined above;

R₆ is selected from the group consisting of H, C₁-C₉ alkyl, C₂-C₉ alkenyl, C₂-C₉ alkynyl, C₃-C₉ carbocyclyl, C₂-C₉ heterocyclyl, C₂-C₉ heteroaryl, C₆-C₁₂ aryl, C₁-C₉ alkyloxy, C₁-C₉ alkylthio, C₆-C₁₂ arylthio, C₁-C₉ hydroxyalkyl, C₁-C₉ alkyloxy, C₆-C₁₂ aryloxy, C₁₋₁₂ carbonyl, C₁-C₉ fluoroalkyl, C₁-C₉ perfluoroalkyl, halogen, —SH, —OH, —N₃, —NH₂, —NO₂, and CN; and

n is 1, 2, 3, or 4.

In a preferred embodiment, compounds of Formula I are selected from the group consisting of compounds 6 to 31 of Table 2; compounds 32 to 51 of Table 3; compounds 52 to 62 of Table 4; compounds 63 to 78 of Table 5; compounds 79 to 92 of Table 6; compounds 93 to 103 of Table 7; compounds 104 to 118 of Table 8; compounds 119 to 122 of Table 9; compounds 123 to 126 of Table 10; compounds 127 to 129 of Table 11; compounds 130 to 132 of Table 12; compounds 133 to 136 of Table 13; compounds 137 to 139 of Table 14; compounds 140 to 142 of Table 15; compounds 143 to 148 of Table 16; compounds 149 to 153 of Table 17; compounds to 154 to 157 of Table 18; compounds 158 to 161 of Table 19; compounds 162 to 169 of Table 20; compounds 170 to 172 of Table 21; and compounds 173 to 182 of Table 22 depicted herein.

More preferably, compounds of Formula (I) are selected from the group consisting of compounds 6, 13, 24, and 25 of Table 2; compounds 33 to 35, 38 to 41, 43, 44, and 47 to 49 of Table 3; compounds 53, 55, and 58 of Table 4; compounds 67, 68, and 72 to 76 of Table 5; compounds 87 and 90 of Table 6; compounds 98 and 103 of Table 7; compounds 106 and 114 of Table 8; compounds 119 and 121 of Table 9; compounds 123 and 125 of Table 10; compounds 127 to 129 of Table 11; compound 130 of Table 12; compounds 133 to 136 of Table 13; compounds 137 and 138 of Table 14; compounds 144 and 146 of Table 16; compound 150 of Table 17; compounds 154 and 156 of Table 18; and compound 167 of Table 20 depicted herein. In a most preferred embodiment, the compounds above are active Nec-5 compounds.

The present invention is also directed to a pharmaceutical composition comprising a compound of Formula (I):

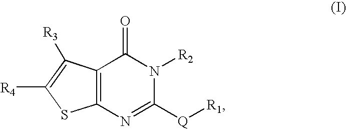

or a pharmaceutically acceptable salt thereof, where Q, R₁, R₂, R₃, and R₄ are defined as above;

and a pharmaceutically acceptable excipient.

In a preferred embodiment, the pharmaceutical composition is comprised of compounds of Formula I selected from the group consisting of compounds 1 and 6 to 31 of Table 2; compounds 32 to 51 of Table 3; compounds 52 to 62 of Table 4; compounds 63 to 78 of Table 5; compounds 79 to 92 of Table 6; compounds 93 to 103 of Table 7; compounds 104 to 118 of Table 8; compounds 119 to 122 of Table 9; compounds 123 to 126 of Table 10; compounds 127 to 129 of Table 11; compounds 130 to 132 of Table 12; compounds 133 to 136 of Table 13; compounds 137 to 139 of Table 14; compounds 140 to 142 of Table 15; compounds 143 to 148 of Table 16; compounds 149 to 153 of Table 17; compounds to 154 to 157 of Table 18; compounds 158 to 161 of Table 19; compounds 162 to 169 of Table 20; compounds 170 to 172 of Table 21; and compounds 173 to 182 of Table 22 depicted herein.

More preferably, the pharmaceutical composition is comprised of compounds of Formula (I) selected from the group consisting of compounds 1 and 6, 13, 24, and 25 of Table 2; compounds 33 to 35, 38 to 41, 43, 44, and 47 to 49 of Table 3; compounds 53, 55, and 58 of Table 4; compounds 67, 68, and 72 to 76 of Table 5; compounds 87 and 90 of Table 6; compounds 98 and 103 of Table 7; compounds 106 and 114 of Table 8; compounds 119 and 121 of Table 9; compounds 123 and 125 of Table 10; compounds 127 to 129 of Table 11; compound 130 of Table 12; compounds 133 to 136 of Table 13; compounds 137 and 138 of Table 14; compounds 144 and 146 of Table 16; compound 150 of Table 17; compounds 154 and 156 of Table 18; and compound 167 of Table 20 depicted herein. In a most preferred embodiment, the pharmaceutical composition is comprised of those compounds above which are active Nec-5 compounds.

The present invention is also directed to a method of synthesizing compounds of Formula (I-A):

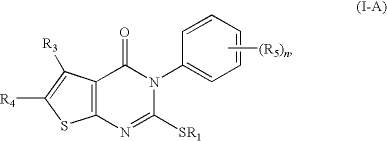

or a pharmaceutically acceptable salt thereof, where

R₁ is selected from the group consisting of H, C₁-C₉ alkyl, C₂-C₉ alkyenyl, C₂-C₉ alkynyl, C₆-C₁₂ aryl, and C₁-C₁₂ carbonyl;

R₂ is selected from the group consisting of C₁-C₉ alkaryl, and C₆-C₁₂ aryl; and

R₃ and R₄ are, independently, selected from the group consisting of C₁-C₉ alkyl, C₂-C₉ alkenyl, C₂-C₉ alkynyl, C₁-C₉alkyloxy, and C₁-C₁₂ carbonyl, or

R₃ and R₄, combined, form an C₃-C₉ carbocyclic, C₂-C₉ heterocyclic, C₆-C₁₂ aryl, or C₂-C₁₂ heteroaryl, ring system;

R₅ is selected from the group consisting of H, C₁-C₉ alkyl, C₂-C₉ alkenyl, C₂-C₉ alkynyl, C₃-C₉ carbocyclyl, C₂-C₉ heterocyclyl, C₂-C₉ heteroaryl, C₆-C₁₂ aryl, C₁-C₉ alkyloxy, C₁-C₉ alkylthio, C₆-C₁₂ arylthio, C₁-C₉ hydroxyalkyl, C₁-C₉ alkyloxy, C₆-C₁₂ aryloxy, C₁₋₁₂ carbonyl, C₁-C₉ fluoroalkyl, C₁-C₉ perfluoroalkyl, halogen, —SH, —OH, —N₃, —NH₂, —NO₂, and CN; and

n is 1, 2, 3, or 4;

where the method comprises providing a compound of Formula (I-B):

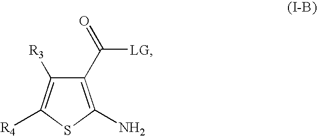

wherein

LG is C₁-C₉ alkyloxy, C₁-C₉ alkylsulfonyloxy, C₆-C₁₂ arylsulfonyloxy, or a halogen; and

reacting the compound of Formula (I-B) with an C₆-C₁₂ aryl isothiocyanate to provide a compound of Formula (I-C):

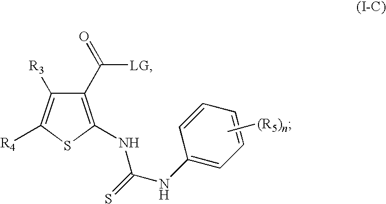

where the compound of Formula (I-C) is thereafter transformed to produce a compound of Formula (I-A).

The present invention is further directed to a method of synthesizing compounds of Formula (I-A), where the compound of Formula (I-B) is obtained from a compound of Formula (I-E):

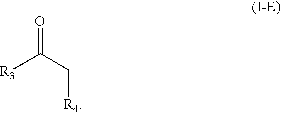

The present invention is also directed to a method of treating a subject with a disease or condition as provided Table 1 comprising administering to a subject an effective amount of a compound of Formula (I), with the proviso that compounds where Q is —S—, R₁ is —CH₂CN, R₂ is —C₆H₄(4-OMe), and R₃ and R₄, combined, form an unsubstituted C₆-carbocyclic six-membered ring, are specifically excluded.

In a preferred embodiment, the compounds of Formula I are selected from the group consisting of compounds 6 to 31 of Table 2; compounds 32 to 51 of Table 3; compounds 52 to 62 of Table 4; compounds 63 to 78 of Table 5; compounds 79 to 92 of Table 6; compounds 93 to 103 of Table 7; compounds 104 to 118 of Table 8; compounds 119 to 122 of Table 9; compounds 123 to 126 of Table 10; compounds 127 to 129 of Table 11; compounds 130 to 132 of Table 12; compounds 133 to 136 of Table 13; compounds 137 to 139 of Table 14; compounds 140 to 142 of Table 15; compounds 143 to 148 of Table 16; compounds 149 to 153 of Table 17; compounds to 154 to 157 of Table 18; compounds 158 to 161 of Table 19; compounds 162 to 169 of Table 20; compounds 170 to 172 of Table 21; and compounds 173 to 182 of Table 22 depicted herein.

More preferably, the compounds of Formula (I) are selected from the group consisting of compounds 6, 13, 24, and 25 of Table 2; compounds 33 to 35, 38 to 41, 43, 44, and 47 to 49 of Table 3; compounds 53, 55, and 58 of Table 4; compounds 67, 68, and 72 to 76 of Table 5; compounds 87 and 90 of Table 6; compounds 98 and 103 of Table 7; compounds 106 and 114 of Table 8; compounds 119 and 121 of Table 9; compounds 123 and 125 of Table 10; compounds 127 to 129 of Table 11; compound 130 of Table 12; compounds 133 to 136 of Table 13; compounds 137 and 138 of Table 14; compounds 144 and 146 of Table 16; compound 150 of Table 17; compounds 154 and 156 of Table 18; and compound 167 of Table 20 depicted herein. In a most preferred embodiment, the pharmaceutical composition is comprised of those compounds above which are active Nec-5 compounds.

The present invention is further directed to a method of treating a subject with a disease or condition as provided Table 1 comprising administering to a subject an effective amount of a pharmaceutical composition of compounds of Formula (I).

In a preferred embodiment, the pharmaceutical composition is comprised of compounds of Formula I selected from the group consisting of compounds 1 and 6 to 31 of Table 2; compounds 32 to 51 of Table 3; compounds 52 to 62 of Table 4; compounds 63 to 78 of Table 5; compounds 79 to 92 of Table 6; compounds 93 to 103 of Table 7; compounds 104 to 118 of Table 8; compounds 119 to 122 of Table 9; compounds 123 to 126 of Table 10; compounds 127 to 129 of Table 11; compounds 130 to 132 of Table 12; compounds 133 to 136 of Table 13; compounds 137 to 139 of Table 14; compounds 140 to 142 of Table 15; compounds 143 to 148 of Table 16; compounds 149 to 153 of Table 17; compounds to 154 to 157 of Table 18; compounds 158 to 161 of Table 19; compounds 162 to 169 of Table 20; compounds 170 to 172 of Table 21; and compounds 173 to 182 of Table 22 depicted herein.

More preferably, the pharmaceutical composition is comprised of compounds of Formula (I) selected from the group consisting of compounds 1 and 6, 13, 24, and 25 of Table 2; compounds 33 to 35, 38 to 41, 43, 44, and 47 to 49 of Table 3; compounds 53, 55, and 58 of Table 4; compounds 67, 68, and 72 to 76 of Table 5; compounds 87 and 90 of Table 6; compounds 98 and 103 of Table 7; compounds 106 and 114 of Table 8; compounds 119 and 121 of Table 9; compounds 123 and 125 of Table 10; compounds 127 to 129 of Table 11; compound 130 of Table 12; compounds 133 to 136 of Table 13; compounds 137 and 138 of Table 14; compounds 144 and 146 of Table 16; compound 150 of Table 17; compounds 154 and 156 of Table 18; and compound 167 of Table 20 depicted herein. In a most preferred embodiment, the pharmaceutical composition is comprised of those compounds above which are active Nec-5 compounds.

These methods of treating a subject are directed to diseases or conditions which include chronic neurodegenerative disease; acute neurological disease; acute neurodegeneration; the result of cell death associated with renal failure; the result of retinal neuronal cell death; the result of cell death of cardiac muscle; the result of cell death of cells of the immune system; myocardial infarction; cardiac infarction; stroke; ischemic stroke; hemorrhagic stroke; ischemia; ischemic liver disease, pancreatic disease, heart disease, brain disease, kidney disease or injury; ischemic mesenteric, retinal, or neuronal injury; ischemic injury during organ storage; delayed ischemic brain injury; traumatic brain injury; head trauma; sepsis; septic shock; necroptosis; necrosis; ischemic necrosis; retinal necrosis; necrotizing myopathy of intensive care; primary systemic infection; pancreatitis; and cell death induced by LPS.

Preferably, chronic neurodegenerative diseases are Alzheimer's disease; Huntington's disease; Parkinson's disease; amyotrophic lateral sclerosis; HIV-associated dementia; cerebral ischemia; amyotropic lateral sclerosis; multiple sclerosis; Lewy body disease; Menke's disease; Wilson's disease; Creutzfeldt-Jakob disease; and Fahr disease.

### DEFINITIONS

By “Nec-5 compound” is meant 3-p-methoxyphenyl-5,6-tetra-methylenothieno-[2,3-d]-pyrimidin-4-one-2-mercaptoethylcyanide (compound 1, Table 2), and structural analogs thereof (for example, compounds 6 to 182 of Tables 2 to 22 described herein) which are encompassed by Formula (I), and which may be encompassed by substructures Formulae (II), (III), or (IV), and further, may be encompassed by substructures Formulae (V) to (VII), (XII) to (XXVIII) or (XXIX), depicted herein.

By an “active Nec-5 compound” is meant a Nec-5 compound, defined above, which decreases necrosis (for example, compounds 1, 6, 13, 24, 25, 33 to 35, 38 to 41, 43, 44, 47 to 49, 53, 55, 58, 67, 68, 72 to 76, 87, 90, 98, 103, 106, 114, 119, 121, 123, 125, 127 to 130, 133 to 138, 144, 146, 150, 154, 156, and 167 of Tables 2 to 14, Tables 16 to 18, and Table 20).

By “decreases necrosis” or “decreasing necrosis” is meant reducing the number of cells which undergo necrosis relative to a control cell receiving a cell death stimulus, such as, for example, by contacting the cell with TNFα or DMSO, without a candidate small molecule inhibitor. Preferably necrosis is decreased 10% relative to a control. More preferably necrosis is decreased 50% relative to a control. Most preferably necrosis is decreased 90% relative to a control. Preferably a decrease in necrosis is tested by determining the ATP level in a cell which has received a candidate compound, such as a compound from a chemical library, and comparing it to the ATP level in a control cell. Necrosis is decreased in a cell treated with a candidate compound in which the ATP level does not decrease as much as it does in the control cell.

By “candidate compound” is meant a chemical, be it naturally-occurring or artificially-derived, that is surveyed for its ability to modulate the level of necrosis by employing one of the assay methods described herein. Candidate compounds may include, for example, peptides, polypeptides, synthesized organic molecules, naturally occurring organic molecules, nucleic acid molecules, and components thereof.

By “cell death” is meant the death of a cell by either apoptosis or necrosis.

By “necrosis” is meant caspase-independent cell death characterized by cellular ATP depletion. Preferably the cell is depleted of ATP 10% relative to a control cell, receiving vehicle only (for example, DMSO). More preferably, the cell is depleted of ATP 50% relative to a control cell. Most preferably, the cell is depleted of ATP 90% relative to a control cell. Preferably, necrosis is tested by determining the ATP level in a cell which has received a compound, for example, zVAD-fmk, DMSO, or TNFα, and comparing it to the ATP level in a cell-receiving vehicle only. Necrosis occurs in a cell treated with a candidate compound in which the ATP level decreases relative to the control cell.

Necrosis may be liquefactive, may affect adipose or hepatic tissue, and may be caseous or fibrinoid. A cell may undergo necrosis in response to ischemic cell injury or viral infection.

By “caspase-independent cell death” is meant cell death that occurs when apoptosis is prevented. Apoptosis may be prevented by contacting a cell with a caspase inhibitor such as zVAD-fmk at a concentration sufficient enough that the cell survives when stimulated to undergo apoptosis, for example, by treatment with an apoptosis-promoting drug or ionizing radiation.

By “apoptosis” is meant cell death characterized by any of the following properties: nuclear condensation, DNA fragmentation, membrane blebbing, or cell shrinkage.

By “modulation of intracellular signaling pathways mediated by TNFα” is meant a change in the communication between components of a cell in response to contacting the cell with TNFα. The change may be in the way or duration in which proteins within the cell interact, or the way or duration in which proteins are altered, such as by phosphorylation or dephosphorylation, or in the way or duration in which proteins interact with DNA.

By “modulation of intracellular signaling pathways mediated by DMSO” is meant a change in the communication between components of a cell in response to contacting the cell with DMSO. The change may be in the way or duration in which proteins within the cell interact, or the way or duration in which proteins are altered, such as by phosphorylation or dephosphorylation, or in the way or duration in which proteins interact with DNA.

The term “subject” is meant a patient in need thereof, and the term “patient” includes any mammal such as a human, a domestic pet or livestock.

By “treating” is meant to administer to a subject, cell, lysate or extract derived from a cell, or a molecule derived from a cell, a compound that decreases necrosis.

By “condition” is meant a state of being or feeling. Conditions include, but are not limited to, those listed in Table 1.

By “effective amount” is meant the amount of a compound required to treat or prevent an infection. The effective amount of active compound(s) used to practice the present invention for therapeutic treatment of conditions of Table 1 caused by or contributed to by necrosis varies depending upon the manner of administration, the age, body weight, and general health of the subject. Ultimately, the attending physician or veterinarian will decide the appropriate amount and dosage regimen. Such amount is referred to as an “effective” amount.

By “neurodegenerative disease” is meant a disease characterized by neuronal cell death. Examples of neurodegenerative diseases include, but are not limited to, Alzheimer's disease, amyotropic lateral sclerosis, cerebral ischemia, Creutzfeldt-Jakob disease, Fahr disease, Huntington's disease and related polyglutamine expansion diseases, Lewy body disease, Menke's disease, multiple sclerosis, stroke, and Wilson's disease.

By “neuron” is meant a cell of ectodermal embryonic origin derived from any part of the nervous system of an animal. Neurons express well-characterized neuron-specific markers which include neurofilament proteins, MAP2, and class III β-tubulin. Included as neurons are, for example, hippocampal, cortical, midbrain dopaminergic, motor, sensory, sympathetic, septal cholinergic, and cerebellar neurons.

By a “dosage sufficient to decrease necrosis” is meant an amount of a chemical compound or small molecule which when administered to a subject will decrease necrosis. Preferably necrosis is decreased in the subject 10% relative to an untreated subject. More preferably necrosis is decreased in the subject 50% relative to an untreated subject. Most preferably necrosis is decreased in the subject 90% relative to an untreated subject.

As used herein, by “measuring necrosis” is meant determining if a cell is dying through necrosis, in the presence of a compound, compared to a cell which is not in the presence of the compound (control cell). Necrosis can be measured by determining cellular ATP levels, wherein a cell that is undergoing necrosis has a decreased level of cellular ATP compared to a control cell. Necrosis may also be measured by staining with a vital dye, for example, trypan blue, wherein a cell which is necrosing will be stained with the vital dye, and a cell which is not necrosing will not be stained with the dye.

The term “pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M Berge et al. describe pharmaceutically acceptable salts in detail in J. Pharmaceutical Sciences, 1977, 66:1-19. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecyl sulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

Compounds useful in the invention include those described herein in any of their pharmaceutically acceptable forms, including acid or base addition salts, solvates, and polymorphs thereof.

By “ischemia” is meant a cardiovascular disorder characterized by a low oxygen state usually due to the obstruction of the arterial blood supply or inadequate blood flow leading to hypoxia in the tissue.

By “myocardial infarction” is meant a cardiovascular disorder characterized by localized necrosis resulting from obstruction of the blood supply.

By “stroke” is meant a cardiovascular disorder caused by a blood clot or bleeding in the brain, most commonly caused by an interruption in the flow of blood in the brain as from clot blocking a blood vessel. In certain embodiments of the invention, the term “stroke” refers to ischemic stroke or hemorrhagic stroke.

By “trauma” is meant any physical damage to the body caused by violence, accident, fracture, etc.

### CHEMICAL DEFINITIONS

In the generic descriptions of compounds of this invention, the number of atoms of a particular type in a substituent group is generally given as a range, e.g., an alkyl group containing from 1 to 9 carbon atoms or C₁₋₉ alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner.

As used herein, the definition of each expression, e.g., R₅, R₆, alkyl, m, n, etc., when it occurs more than once in any structure, is intended to be independent of its definition elsewhere in the same structure.

For purposes of this invention, heteroatoms such as nitrogen and sulfur may have hydrogen or alkyl substituents and/or any permissible substituents of organic compounds described herein which satisfy the valences of the heteroatoms. This invention is not intended to be limited in any manner by the permissible substituents of organic compounds.

In any of the following definitions, the terms “optionally substituted,” “substitution,” or “substituted,” includes the proviso that such substitution is in accordance with permitted valence of the substituted atom and the substituent, and that the substitution results in a stable compound, e.g., which does not spontaneously undergo transformation such as by rearrangement, cyclization, elimination, etc. The terms “optionally substituted,” “substitution,” or “substituted,” includes all permissible substituents of organic compounds. Illustrative substituents can be one or more and the same or different for appropriate organic compounds, as for example, alkyl; alkenyl; alkynyl; carbocyclyl (e.g., cycloalkyl; cycloalkenyl); heterocyclyl, heteroaryl, aryl; hydroxy (—OH); halogen (F, Cl, Br, I); azido (—N₃); nitro (—NO₂); oxo (═O); imino (═NH); cyano (—CN); fluoroalkyl (e.g., —CH₂F); perfluoroalkyl (e.g., CF₃), hydroxyalkyl (—(R_{A})OH)); alkyloxy (—OR_{B}); aryloxy (—OR_{C}); thio (—SH), alkylthio (—SR_{D}); sulfonyl (—SO₂R_{F}), arylthio (—SR_{I}); carbonyl(amides (—C(O)NH₂ or —C(O)NR_{J}R_{K}), ketones (—C(O)R_{L}), aldehydes (—C(O)H or CHO), esters (e.g., —OC(O)R_{M}, or —CO₂R_{N}), carboxylic acids (—C(O)OH)); amino (—NH₂ or NR_{O}R_{P}); and sulfinyl (—S(O)R_{Q}); where each of R_{A}, R_{B}, R_{C}, R_{D}, R_{E}, R_{F}, R_{G}, R_{H}, R_{I}, R_{J}, R_{K}, R_{L}, R_{M}, R_{N}, R_{O}, R_{P}, and R_{Q}, is, independently, selected from the illustrative substituents as defined above, and which also include H. The terms “optionally substituted,” “substitution,” or “substituted,” also include substitution on an aryl or phenyl ring, and include, for example, di- (e.g., ortho-, meta-, para-), tri-, and tetra-substitution. The terms “ortho, meta, and para” apply to 1,2-, 1,3- and 1,4-disubstituted benzenes, respectively. For example, the names 1,2-dimethylbenzene and ortho-dimethylbenzene are synonymous.

As used herein, the terms “alkyl” and the prefix “alk-” are inclusive of both straight chain and branched chain groups and of mono- or polycyclic groups (i.e., cycloalkyl), and may be optionally substituted or unsubstituted. If not specified, alkyl means C₁₋₉ alkyl, i.e., a group with 1 to 9 carbon atoms. Exemplary cyclic groups include cyclopropyl, cyclobutyl, cyclopentyl, cyclohexyl, and cyclohexyl groups. C₁₋₉ alkyls include, without limitation, methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; n-hexyl, cyclohexyl, n-octyl, cyclooctyl, n-nonyl, and cyclononyl.

The terms “alkenyl” and “alkynyl” refer to unsaturated aliphatic groups analogous in length and possible substitution to the alkyls described above, but that contain at least one double or triple bond respectively.

By “C₂₋₉ alkenyl” are inclusive of both straight chain and branched chain groups and of mono- or polycyclic groups (i.e., cycloalkenyl), containing one or more double bonds, and may be optionally substituted or unsubstituted. If not specified, alkyenyl means C₂₋₉ alkenyl, i.e., a group with 2 to 9 carbon atoms. C₂₋₉ alkenyls include, without limitation, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl 2,2-dimethyl-3-butenyl; 1-pentenyl; cyclopentenyl; 1-hexenyl; cyclohexenyl; 1-heptenyl; cycloheptenyl; 1-octenyl; cyclooctenyl; 1-nonenyl; and cyclononenyl.

By “C₂₋₉ alkynyl” are inclusive of both straight chain and branched chain groups containing one or more triple bonds, and may be optionally substituted or unsubstituted. If not specified, alkynyl means C₂₋₉ alkynyl, i.e., a group with 2 to 9 carbon atoms. C₂₋₇ alkynyls include, without limitation, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butynyl; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl; 3-methyl-4-pentynyl; 1-hexynyl; 1-heptynyl; 1-octynyl; and 1-nonynyl.

By “aromatic” is meant a (4n+2) Hückel ring system, i.e. a fully conjugated ring system with (4n+2) π electrons, wherein the sum of (4n+2) equals the number of electron pairs, and n is a whole number.

By “non-aromatic” is meant a saturated or unsaturated ring system which is not aromatic [(4n+2) Hückel] or anti-aromatic [(4n) anti-Hückel].

By “C₃₋₉ carbocyclic” or “C₃₋₉ carbocyclyl” is meant a non-aromatic ring system, saturated or unsaturated, consisting of all carbon ring atoms, and are inclusive of both C₃₋₉ cycloalkyl and C₃₋₉ cycloalkenyl groups. The carbocyclic ring may be covalently attached or fused to another ring via any carbon atom to provide a stable bicyclic structure, and may be optionally substituted or unsubstituted. If not specified, “carbocyclic” or “carbocyclyl” means C₃₋₉ carbocyclic or C₃₋₉ carbocyclyl, i.e., a ring with 3 to 9 carbon atoms.

By “C₂₋₉ heterocyclic” or “C₂₋₉ heterocyclyl” is meant a 5- to 7-membered monocyclic or 7- to 14-membered bicyclic ring system which is saturated or unsaturated, but which is not aromatic, and which consists of 2 to 9 carbon atoms and 1, 2, 3 or 4 heteroatoms independently selected from the group consisting of N, O, and S. If not specified, “heterocyclic” or “heterocyclyl” means a C₂₋₉ heterocyclic or C₂₋₉ heterocyclyl, i.e., containing 2 to 9 carbon atoms. The heterocyclyl group may be optionally substituted or unsubstituted. The nitrogen and sulfur heteroatoms may optionally be oxidized. The heterocyclyl ring may be covalently attached or fused via any heteroatom or carbon atom to another ring provide a stable bicyclic ring structure. Exemplary heterocyclyls include, without limitation, aziridinyl, azetidinyl, pyrrolidinyl, piperidinyl, piperazinyl, morpholinyl, azocanyl, thiacyclohexyl, thiocyclopentyl, oxiranyl, 1,3-dioxacyclopentanyl, 1,3-dioxanyl, 1,4-dioxanyl, 1,3-dithiolanyl, 1,4-dithiolanyl tetrahydrofuranyl, tetrahydroisoquinolinyl, and tetrahydroquinolinyl.

By “C₂₋₉ heteroaromatic” or “C₂₋₉ heteroaryl” is meant an aromatic [(4n+2) Hückel] ring system consisting of both carbon ring atoms and hetero-ring atoms (e.g., N, O, or S). “C₂₋₉ heteroaryl” is meant a stable 5- to 7-membered monocyclic or 7- to 14-membered bicyclic ring which is unsaturated (heteroaromatic), and which consists of 2 to 9 carbon atoms and 1, 2, 3 or 4 heteroatoms independently selected from the group consisting of N, O, and S, and includes any bicyclic group fused to a benzene ring. If not specified, “heteroaromatic” or “heteroaryl” means a C₂₋₉ heteroaryl, i.e., a group with 2 to 9 carbon atoms, and may be optionally substituted or unsubstituted. The nitrogen and sulfur heteroatoms may optionally be oxidized. The heteroaryl ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Exemplary heteroaryls include, without limitation, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, dithiazinyl, furanyl, furazanyl, imidazolidinyl, imidazolinyl, imidazolyl, indolenyl, indolinyl, indolizinyl, indolyl, isobenzofuranyl, isoindazolyl, isoindolinyl, isoindolyl, isoquinolinyl, isothiazolyl, isoxazolyl, naphthyridinyl, phenanthridinyl, phenanthrolinyl, phenarsazinyl, phenazinyl, phenothiazinyl, phenoxathiinyl, phenoxazinyl, phthalazinyl, pteridinyl, pyranyl, pyrazinyl, pyrazolidinyl, pyrazolinyl, pyrazolyl, pyridazinyl, pyridooxazole, pyridoimidazole, pyridothiazole, pyridinyl, quinazolinyl, quinolizinyl, quinoxalinyl, and xanthenyl.

By “C₆₋₁₂ aryl” is meant an aromatic group having a ring system comprised of carbon atoms with conjugated π electrons (e.g., phenyl). If not specified, “aryl” means a C₆₋₁₂ aryl, i.e., an aromatic, mono- or bicyclic ring system with 6 to 12 carbon atoms, which may be optionally substituted or unsubstituted.

By “fluoroalkyl” is meant a C₁₋₉ alkyl group which is substituted with hydrogen, and one or more fluorine atoms.

By “perfluoroalkyl” is meant a C₁₋₉ alkyl group consisting of only carbon and fluorine atoms.

By “hydroxyalkyl” is meant a chemical moiety with the formula —(R_{A})—OH, wherein R_{A} is an optionally substituted C₁₋₉ alkyl group as defined herein.

By “alkoxy” or “alkyloxy” is meant a chemical substituent of the formula OR_{B}, wherein R_{B} is an optionally substituted C₁₋₉ alkyl group as defined herein.

By “aryloxy” is meant a chemical substituent of the formula OR_{S}, wherein R_{C} is an optionally substituted C₆₋₁₂ aryl group as defined herein.

By “alkylthio” is meant a chemical substituent of the formula SR_{D}), wherein R_{D} is an optionally substituted C₁₋₉ alkyl group as defined herein.

By “sulfoxide” is meant S(O)R_{E}, wherein R_{E} is an optionally substituted C₁₋₉ alkyl or C₆₋₁₂ aryl group as defined herein.

By “sulfonyl” is meant —SO₂R_{F} wherein R_{F} is an optionally substituted C₁₋₉ alkyl (to form a “C₁₋₉ alkylsulfonyl”) or C₆₋₁₂ aryl (to form a “C₆₋₁₂ arylsulfonyl”), or an amino group, as defined herein.

By “alkylsulfonyloxy” is meant —OSO₂R_{G}, wherein R_{G} is an optionally substituted C₁₋₉ alkyl group as defined herein. An exemplary alkylsulfonyloxy group includes an O-mesyl (—OMs or —O-(methanesulfonyl)) group.

By “arylsulfonyloxy” is meant —OSO₂R_{B}, wherein R_{H} is an optionally substituted C₆₋₁₂ aryl group, as defined herein. An exemplary arylsulfonyloxy group includes an O-tosyl group (—OTs or —O-(toluenesulfonyl)).

By “arylthio” is meant —SR₁, wherein R₁ is an C₆₋₁₂ aryl group as defined herein.

By “C₁₋₁₂ carbonyl” is meant amides (—C(O)NH₂ or —C(O)NR_{J}R_{K}), ketones (—C(O)R_{L}), aldehydes (—C(O)H or —CHO), esters (e.g., —OC(O)R_{M}, or —CO₂R_{N}), carboxylic acids (—C(O)OH), and the like, wherein R_{J}, R_{K}, R_{L}, R_{M}, and R_{N}, are optionally substituted C₁₋₉ alkyl or optionally substituted C₆₋₁₂ aryl, as defined herein.

By “amino” is meant —NH₂ or —NR_{O}R_{P}, wherein R_{O} and R_{P} are optionally substituted C₁₋₉ alkyl or C₆₋₁₂ aryl, as defined herein.

By “aryl isothiocyanate,” is meant Ar—N═C═S, wherein the C₆₋₁₂ aryl group (i.e., Ar) may be further optionally substituted or unsubstituted, as defined herein.

By “halogen,” “halide,” or “halo,” is meant —F, —Cl, —Br or —I; by “sulfhydryl” or “thio” is meant —SH; and by “hydroxyl” is meant —OH.

Contemplated equivalents of the compounds described above include compounds which otherwise correspond thereto, and which have the same general properties thereof (e.g., functioning as inhibitors of cellular necrosis), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants, which are known by one skilled in the art, but are not mentioned here.

For purposes of this invention, the chemical elements are identified in accordance with the Periodic Table of the Elements, CAS version, Handbook of Chemistry and Physics, 67th Ed., 1986-87, inside cover.

Other features and advantages of the invention will be apparent from the following detailed description and from the claims.

### DETAILED DESCRIPTION OF THE INVENTION

Described herein are compounds, pharmaceutical compositions, methods of synthesis, and methods for treating a range of conditions. While this application focuses on conditions in which cell or tissue necrosis is a causative factor or result, any condition listed in Table 1 may be treated using the compounds, compositions, and methods of the invention. Techniques for making and using the invention are now described in detail.

### Compounds

The present invention is directed to compounds, or their pharmaceutically acceptable salts, encompassed by Formula (I), in which

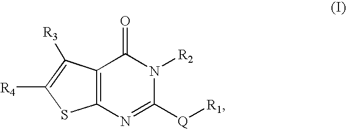

Q is —S—, —S(O)—, or —S(O)₂—;

R₁ is a C₁-C₉ alkyl, C₂-C₉ alkyenyl, C₂-C₉ alkynyl, C₆-C₁₂ aryl, or a C₁-C₁₂ carbonyl; R₂ is a C₁-C₉ alkaryl or a C₆-C₁₂ aryl; R₃ and R₄ are C₁-C₉ alkyl, C₂-C₉ alkenyl, C₂-C₉ alkynyl, C₁-C₉ alkyloxy, or C₁-C₁₂ carbonyl, or R₃ and R₄, combined, form a C₃-C₉ carbocyclic, C₂-C₉ heterocyclic, C₆-C₁₂ aryl, or C₂-C₁₂ heteroaryl, ring system;

with the proviso that compound (1), chemically known as 3-p-methoxyphenyl-5,6-tetra-methylenothieno-[2,3-d]-pyrimidin-4-one-2-mercaptoethylcyanide (where Q is —S—, R₁ is —CH₂CN, R₂ is —C₆H₄(4-OMe), and R₃ and R₄, combined, form an unsubstituted C₆-carbocyclic six-membered ring), as depicted in the Examples, in Table 2, and as depicted below, is

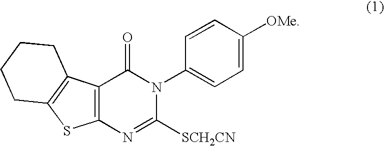

Preferably, compounds of the above Formula (I) may correspond, as well, to substructures Formulae (II), (III), and/or (IV), as depicted below:

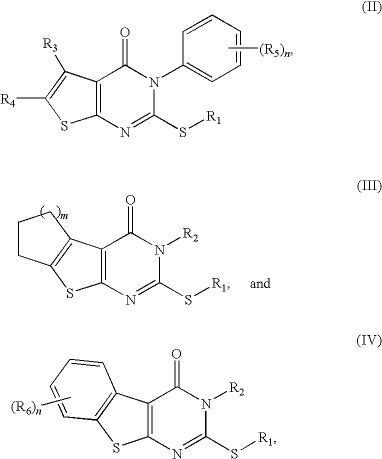

in which R₅ and R₆ are selected from the group consisting of H, C₁-C₉ alkyl, C₂-C₉ alkenyl, C₂-C₉ alkynyl, C₃-C₉ carbocyclyl, C₂-C₉ heterocyclyl, C₂-C₉ heteroaryl, C₆-C₁₂ aryl, C₁-C₉ alkyloxy, C₁-C₉ alkylthio, C₆-C₁₂ arylthio, C₁-C₉ hydroxyalkyl, C₁-C₉ alkyloxy, C₆-C₁₂ aryloxy, C₁₋₁₂ carbonyl, C₁-C₉ fluoroalkyl, C₁-C₉ perfluoroalkyl, halogen, —SH, —OH, —N₃, —NH₂, —NO₂, and —CN;

n is 1, 2, 3, or 4; and m is 1, 2, or 3.

Preferably, these compounds of Formula (I), which may be encompassed by substructures (II), (III), and (IV), are compounds 6-182 depicted in the Examples on Tables 2 through 22. Most preferably, these compounds are active Nec-5 compounds, i.e., compounds 6, 13, 24, 25, 33 to 35, 38 to 41, 43, 44, 47 to 49, 53, 55, 58, 67, 68, 72 to 76, 87, 90, 98, 103, 106, 114, 119, 121, 123, 125, 127 to 130, 133 to 138, 144, 146, 150, 154, 156, and 167.

Compounds of the present invention may be encompassed by substructure Formulae (V) to (VII), (XII) to (XXVIII) or (XXIX) which are depicted below and in the Examples.

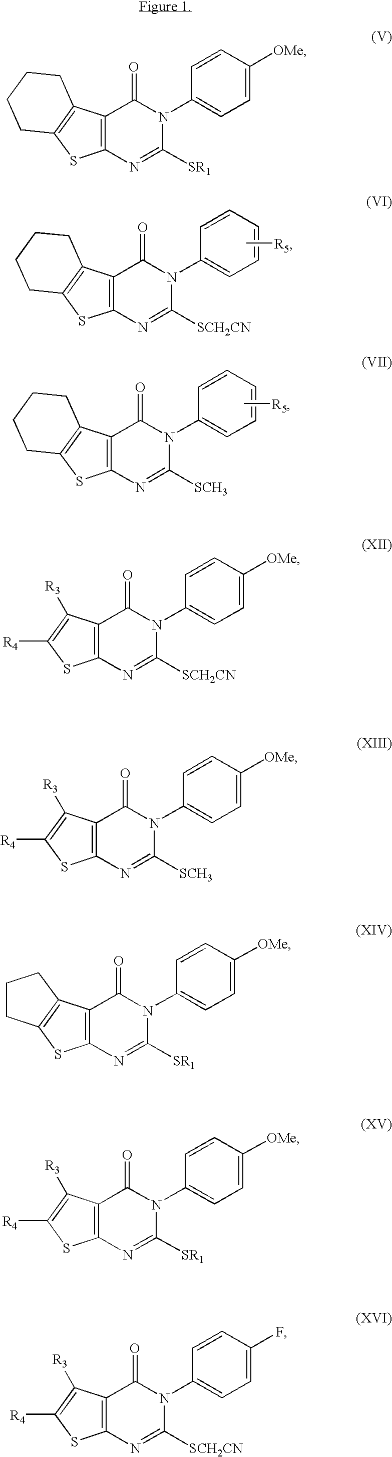

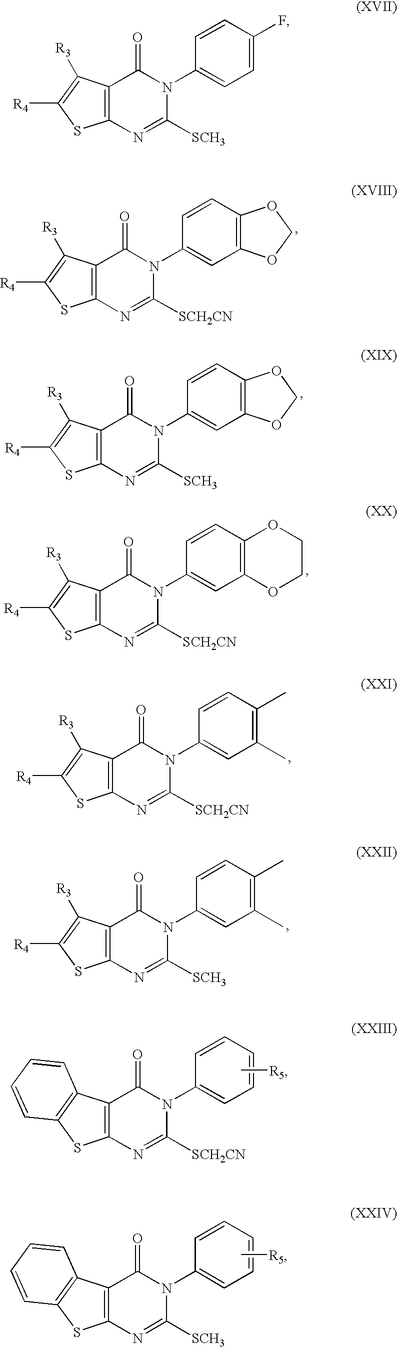

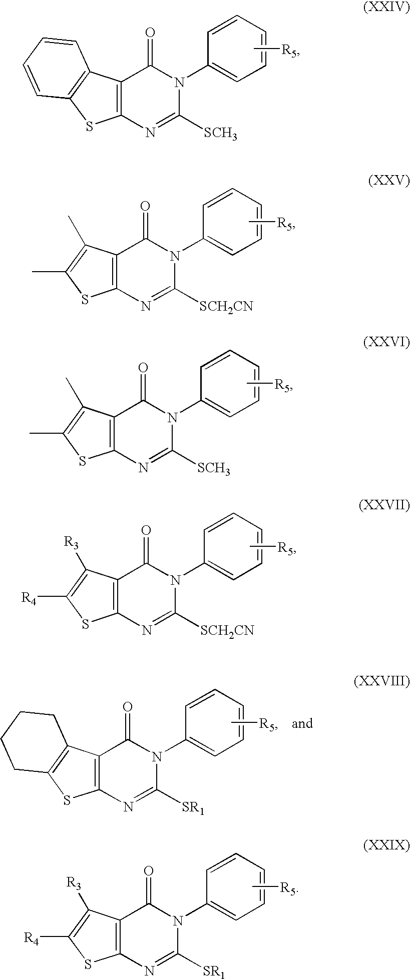

The present invention is also directed to pharmaceutical compositions of compounds of Formula (I), including compound 1, chemically known as 3-p-methoxyphenyl-5,6-tetra-methylenothieno-[2,3-d]-pyrimidin-4-one-2-mercaptoethylcyanide, as depicted in the Examples, and in Table 2, and a pharmaceutically acceptable excipient. Preferably, these compounds include compounds 1, and 6 through 182 depicted in the Examples on Tables 2 through 22. Most preferably, these compounds are active Nec-5 compounds, i.e., compounds 1, 6, 13, 24, 25, 33 to 35, 38 to 41, 43, 44, 47 to 49, 53, 55, 58, 67, 68, 72 to 76, 87, 90, 98, 103, 106, 114, 119, 121, 123, 125, 127 to 130, 133 to 138, 144, 146, 150, 154, 156, and 167.

Any of the compounds or pharmaceutical compositions of the invention can be used together with a set of instructions, i.e., to form a kit.

The present invention is also directed to a method of synthesis of compounds of Formula (I-A), as depicted in Scheme 1, and as detailed in the Examples. Specifically, a compound of Formula (I-A) may be generated starting from a compound of Formula (I-B) (in which LG is a leaving group and can be C₁-C₉ alkyloxy, C₁-C₉ alkylsulfonyloxy, C₆-C₁₂ arylsulfonyloxy, or a halogen). Reaction of a compound of Formula (I-B) with an aryl isothiocyanate, optionally substituted, provides a compound of Formula (I-C), which, upon treatment with ethanolic HCl, smoothly cyclizes to a compound of Formula (I-A) (R₁=H). A compound of Formula (I-A), in which R₁=H, may be further treated with an alkylating agent to form alkylated compounds of Formula (I-A). For example, a compound of Formula (I-A), in which R₁=H, may be treated with methyl iodide (MeI), or 1,1-bromocyanomethane (BrCH₂CN) to form compounds of Formula (I-A) where R₁ is —CH₃ or CH₂CN, respectively.

The present invention is additionally directed to the synthesis of a compound of Formula (I-B) from a ketone compound of Formula (I-E), as depicted in Scheme 1, and as detailed in the Examples. Treatment of appropriately substituted ketone compounds with cyanoacetate, with S₈ and base in refluxing ethanol efficiently generates compounds of Formula (I-B).

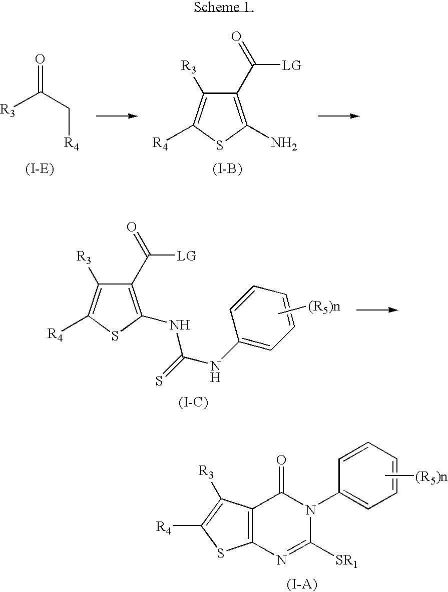

Furthermore, the present invention is directed to a method of treating a subject with a disease or condition, as provided in Table 1, with an effective amount of a compound of Formula (I), as defined herein. Additionally, the present invention is directed to a method of treating a subject with a disease or condition, as provided in Table 1, with an effective amount of a pharmaceutical composition of a compound of Formula (I), as defined herein, and a pharmaceutically acceptable excipient.

TABLE 1

abscess
a condition comprising cell death
associated with renal failure
a condition comprising cell death of
cardiac muscle
a condition comprising cell death of
cells of the immune system
a condition comprising retinal
neuronal cell death
activation-induced cell death
acute neurodegeneration
acute, latent, or persistent viral
infection
acute neurological disease
adenovirus infection
ague
AIDS and associated conditions
alteration of blood vessels
Alzheimer's disease
amyotrophic lateral sclerosis
anemia
ankylosis
anoxia
anthrax lethal toxin induced septic
shock
apnea
arthritis
aspergillosis
asphyxiation
asthma
ataxia
atrophy
avascular necrosis
avascular necrosis of the bone
backache
Becker's muscular dystrophy
blastomycosis
bleeding
blennorhea
bone avascular necrosis
cachexia
cancer
candidiasis (allergic, cutaneous,
mucocutaneous, or systemic)
cardiac infarction
cardiomyopathy
caries
cell death induced by LPS
cerebral ischemia
chemical imbalance
chromoblastomycosis
chronic neurodegenerative diseases
chronic obstructive pulmonary disease
coccidioidomycosis
colic
condition leading to cell or tissue
death
congestive heart failure
constipation
convulsion
coughing
coronary heart disease
Creutzfeldt-Jakob disease
Crohn's disease
cryptococcosis
cyanosis
cytomegalovirus infection
degenerative disease
delayed ischemic brain injury
dementia
diabetes
diarrhea
dizziness
dropsy
dry gangrene
Duchenne muscular dystrophy
dysentery
dyspepsia
dyspnea
edema
emaciation
Epstein-Barr virus infection
facioscapulohumeral muscular
dystrophy
Fahr disease
fainting
fatigue
fever
fibrillation
fungal eye, hair, nail, or skin infection
gangrene
gas gangrene
gastrointestinal disease
genetic disease
graft-versus-host disease
head trauma
hemorrhagic stroke
hepatitis virus infection
Hepatitis B
Hepatitis C
herpes simplex virus infection
high blood pressure
histoplasmosis
HIV-associated dementia
HIV infection and associated
conditions
human herpesvirus infection
human papillomavirus infection
human T-Cell leukemia virus infection
Huntington's disease
hydrops
hypertension
hypotension
icterus
immunodeficiency
indigestion
infection
infectious encephalopathy
insomnia
interruption of blood supply
ischemia
ischemic brain disease or injury
ischemic disease or injury
ischemic heart disease or injury
ischemic injury due to organ storage
ischemic kidney disease or injury
ischemic liver disease or injury
ischemic mesenteric injury
ischemic necrosis
ischemic neuronal injury
ischemic retinal injury
ischemic stroke
itching
jaundice
kidney disease
lack of nutrient or oxygen supply
Landouzy-Dejerine muscular
dystrophy
Lewy body disease
limb-girdle muscular dystrophy
liver cirrhosis
liver disease
liver fibrosis
lobomycosis
low blood pressure
lumbago
lupus
marasmus
measles virus infection
meningitis
Menkes disease
moist gangrene
multifactorial disease (e.g., HIV
infection with opportunistic fungal
infection)
multiple sclerosis
muscle wasting
muscular dystrophy
mycetoma
mycotic keratitis
myocardial infarction
myotonia congenita
myotonic dystrophy
necroptosis
necrosis
necrotic ulceration
necrotizing myopathy of intensive care
neurodegenerative disease
neurological disease
noma
oculomycosis (endogenous or
extension)
onychomycosis
opportunistic infection
otomycosis
pain
pancreatic disease
pancreatitis (chronic, acute, sterile
acute necrotizing, and infected acute
necrotizing)
papovavirus (JC or BK) infection
paracoccidioidomycosis
paralysis
Parkinson's disease
parvovirus infection
penicilliosis
phaeohyphomycosis
physical trauma
piedra
pityriasis versicolor
poisoning
polyglutamine expansion disease
Pompe's disease
primary systemic infection
pruritus
radiation illness
rash
retinal necrosis
rheum
rhinosporidioisis
sclerosis
seizure
sepsis
septic shock
shock
sickle cell disease
skin eruption
sore
spasm
sphacelation
sphacelus
sporotrichosis
Steinert's disease
stroke
superficial infection
systemic infection
tabes
tachycardia
Thomsen's disease
tinea barbae
tinea capitis
tinea corporis
tinea cruris
tinea favosa
tinea nigra
tinea unguium
tooth decay
trauma
traumatic brain injury
tuberculosis
tumor
ulcerative colitis
upset stomach
Varicella-Zoster virus infection
vertigo
viral infection
vomiting
wasting
Wilson's disease
zygomycosis

### Therapy

Therapy according to the invention may be performed alone or in conjunction with another therapy, and may be provided at home, the doctor's office, a clinic, a hospital's outpatient department, or a hospital. Any of the conditions listed in Table 1, alone or present in combination, can be treated using the compounds, compositions, and methods of the invention. Treatment generally begins at a hospital so that the doctor can observe the therapy's effects closely and make any adjustments that are needed. The duration of the therapy depends on the age and condition of the patient, as well as how the patient responds to the treatment. Additionally, a person having a greater risk of developing a condition listed in Table 1 may receive prophylactic treatment to inhibit or delay symptoms of the disease.

Any of the compounds described herein can be used to treat any of the conditions listed in the above Table 1.

Exemplary neurodegenerative diseases are Alzheimer's disease, Huntington's disease, Parkinson's disease, amyotrophic lateral sclerosis, HIV-associated dementia, cerebral ischemia, amyotropic lateral sclerosis, multiple sclerosis, Lewy body disease, Menke's disease, Wilson's disease, Creutzfeldt-Jakob disease, and Fahr disease.

Exemplary muscular dystrophies or related diseases are Becker's muscular dystrophy, Duchenne muscular dystrophy, myotonic dystrophy, limb-girdle muscular dystrophy, Landouzy-Dejerine muscular dystrophy, facioscapulohumeral muscular dystrophy (Steinert's disease), myotonia congenita, Thomsen's disease, and Pompe's disease.

Muscle wasting can be associated with cancer, AIDS, congestive heart failure, and chronic obstructive pulmonary disease, as well as include necrotizing myopathy of intensive care.

Conditions in which alteration in cell proliferation, differentiation or intracellular signaling is a causative factor include cancer and infection, e.g., by viruses (e.g., acute, latent and persistent), bacteria, fungi, or other microbes.

Exemplary viruses are human immunodeficiency virus (HIV), Epstein-Barr virus (EBV), cytomegalovirus (CMV), human herpes viruses (HHV), herpes simplex viruses (HSV), human T-Cell leukemia viruses (HTLV), Varicella-Zoster virus (VZV), measles virus, papovaviruses (JC and BK), hepatitis viruses, adenovirus, parvoviruses, and human papillomaviruses.

In a preferred embodiment, the compounds and methods of the invention can be used to treat any of the following diseases or conditions: chronic neurodegenerative disease; acute neurological disease; acute neurodegeneration; the result of cell death associated with renal failure; the result of retinal neuronal cell death; the result of cell death of cardiac muscle; the result of cell death of cells of the immune system; mycocardial infarction; cardiac infarction; stroke; hemorrhagic stroke; ischemia; ischemic liver disease, pancreatic disease, heart disease, brain disease, kidney disease or injury; ischemic mesenteric, retinal, or neuronal injury; ischemic injury during organ storage; delayed ischemic brain injury; traumatic brain injury; head trauma; sepsis; septic shock; necroptosis; necrosis; ischemic necrosis; retinal necrosis; necrotizing myopathy of intensive care; primary systemic infection; pancreatitis; or cell death induced by LPS.

Compounds and methods of the invention can additionally be used to boost the immune system, whether or not the patient being treated has an immuno-compromising condition. For example, a compound of the present invention can be used in a method to strengthen the immune system during immunization, e.g., by functioning as an adjuvant, or by being combined with an adjuvant.

### Administration of Pharmaceutical Compositions and Formulations

Pharmaceutical compositions and formulations can be prepared utilizing compounds of the invention. Pharmaceutical compositions of the invention are prepared in a manner known to those skilled in the art, for example, by means of conventional dissolving, lyophilising, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

A compound identified as capable of treating any of the conditions of Table 1, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to patients suffering from a disease in which necrosis occurs. Administration may begin before the patient is symptomatic.

Any appropriate route of administration may be employed. For example, the therapy may be administered either directly to the site of a predicted cell death event (for example, by injection) or systemically (for example, by any conventional administration technique). Administration of the compound may also be parenteral, intravenous, intraarterial, subcutaneous, intramuscular, intracranial, intraorbital, ophthalmic, intraventricular, intracapsular, intraspinal, intracisternal, intraperitoneal, intranasal, aerosol, by suppositories, or oral administration. Therapeutic formulations may be in the form of liquid solutions or suspensions; for oral administration, formulations may be in the form of tablets or capsules; and for intranasal formulations, in the form of powders, nasal drops, or aerosols. The dosage of the therapeutic compounds in a pharmaceutically-acceptable formulation depends on a number of factors, including the size and health of the individual patient. The dosage to deliver may be determined by one skilled in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds that decrease necrosis include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

### Dosage

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts. A daily, weekly, or monthly dosage (or other time interval) can be used.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In general, a suitable daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight.

### Combination Therapy

If desired, treatment with compounds of the invention can be combined with therapies for the treatment of any of the conditions of Table 1, e.g., conditions involving necrosis or ischemia. Such treatments include surgery, radiotherapy, chemotherapy, or the administration of one or more additional compounds. Exemplary compounds suitable for combination therapy with compounds of the invention are described below. For example, if desired, treatment with a compound of the invention may be combined with more traditional therapies for a disease characterized by cell death, such as tacrine hydrochloride for the treatment of Alzheimer's disease, or interferon α-1a for the treatment of multiple sclerosis.

Compounds of the invention can be administered in combination with compounds that are apoptosis inhibitors, i.e., compounds that inhibit apoptosis, including but not limited to reversible and irreversible caspase inhibitors. An example of an apoptosis inhibitor includes zVAD (N-benzyloxycarbonyl-Val-Ala-Asp-(OMe) fluoromethyl ketone), IETD (N-acetyl-Ile-Glu-Thr-Asp-al), YVAD (N-benzyloxycarbonyl-Tyr-Val-Ala-Asp-(OMe) fluoromethyl ketone), DEVD (N-[2-(6-hydroxy-3-oxo-3H-xanthen-9-yl)benzoyl]L-α-aspartyl-L-α-glutamyl-N-[(1S)-1-(carboxymethyl)-3-fluoro-2-oxopropyl]-L-Valinamide), and LEHD (N-acetyl-Leu-Glu-His-Asp-al).

In some instances, the compounds of the invention are administered in combination with PARP poly(ADP-ribose) polymerase inhibitors. Non-limiting examples of PARP inhibitors include 6(5H)-Phenanthridinone, 4-Amino-1,8-naphthalimide, 1,5-Isoquinolinediol, and 3-Aminobenzamide.

Compounds of the invention can also be administered in combination with Src inhibitors. Src proteins are mammalian cytoplasmic tyrosine kinases that play an extensive role in signal transduction. Examples of Src inhibitors include but are not limited to: PP1 (1-(1,1-dimethylethyl)-1-(4-methylphenyl)-1H-pyrazolo[3,4-d]pyrimidin-4-amine), PP2 (3-(4-chlorophenyl)-1-(1,1-dimethylethyl)-1H-pyr-azolo[3,4-d]pyrimidin-4-amine), damnacanthal (3-hydroxy-1-methoxy-2-anthra-quinonecarboxaldehyde), and SU-5565.

The methods of the invention involve, in some aspects, combinations of compounds that are inhibitors of cellular necrosis (e.g., heterocyclic thiohydantoin, hydantoin, oxazolidinone, thioxo-oxazolidinone, pyrimidinone, or oxazinanone compounds, or combinations thereof) with agents for the treatment of cardiovascular disorders. Such agents include anti-inflammatory agents, anti-thrombotic agents, anti-platelet agents, fibrinolytic agents, lipid reducing agents, direct thrombin inhibitors, glycoprotein IIb/IIIa receptor inhibitors, agents that bind to cellular adhesion molecules and inhibit the ability of white blood cells to attach to such molecules (e.g. anti-cellular adhesion molecule antibodies), calcium channel blockers, beta-adrenergic receptor blockers, cyclooxygenase-2 inhibitors, angiotensin system inhibitors, and any combinations thereof. One preferred agent is aspirin.

Anti-inflammatory agents include alclofenac; alclometasone dipropionate; algestone acetonide; alpha amylase; amcinafal; amcinafide; amfenac sodium; amiprilose hydrochloride; anakinra; anirolac; anitrazafen; apazone; balsalazide disodium; bendazac; benoxaprofen; benzydamine hydrochloride; bromelains; broperamole; budesonide; carprofen; cicloprofen; cintazone; cliprofen; clobetasol propionate; clobetasone butyrate; clopirac; cloticasone propionate; cormethasone acetate; cortodoxone; deflazacort; desonide; desoximetasone; dexamethasone dipropionate; diclofenac potassium; diclofenac sodium; diflorasone diacetate; diflumidone sodium; diflunisal; difluprednate; diftalone; dimethyl sulfoxide; drocinonide; endrysone; enlimomab; enolicam sodium; epirizole; etodolac; etofenamate; felbinac; fenamole; fenbufen; fenclofenac; fenclorac; fendosal; fenpipalone; fentiazac; flazalone; fluazacort; flufenamic acid; flumizole; flunisolide acetate; flunixin; flunixin meglumine; fluocortin butyl; fluorometholone acetate; fluquazone; flurbiprofen; fluretofen; fluticasone propionate; furaprofen; furobufen; halcinonide; halobetasol propionate; halopredone acetate; ibufenac; ibuprofen; ibuprofen aluminum; ibuprofen piconol; ilonidap; indomethacin; indomethacin sodium; indoprofen; indoxole; intrazole; isoflupredone acetate; isoxepac; isoxicam; ketoprofen; lofemizole hydrochloride; lomoxicam; loteprednol etabonate; meclofenamate sodium; meclofenamic acid; meclorisone dibutyrate; mefenamic acid; mesalamine; meseclazone; methylprednisolone suleptanate; morniflumate; nabumetone; naproxen; naproxen sodium; naproxol; nimazone; olsalazine sodium; orgotein; orpanoxin; oxaprozin; oxyphenbutazone; paranyline hydrochloride; pentosan polysulfate sodium; phenbutazone sodium glycerate; pirfenidone; piroxicam; piroxicam cinnamate; piroxicam olamine; pirprofen; prednazate; prifelone; prodolic acid; proquazone; proxazole; proxazole citrate; rimexolone; romazarit; salcolex; salnacedin; salsalate; salycilates; sanguinarium chloride; seclazone; sermetacin; sudoxicam; sulindac; suprofen; talmetacin; talniflumate; talosalate; tebufelone; tenidap; tenidap sodium; tenoxicam; tesicam; tesimide; tetrydamine; tiopinac; tixocortol pivalate; tolmetin; tolmetin sodium; triclonide; triflumidate; zidometacin; glucocorticoids; and zomepirac sodium.

Anti-thrombotic and fibrinolytic agents include plasminogen (to plasmin via interactions of prekallikrein, kininogens, factors XII, XIIIa, plasminogen proactivator, and tissue plasminogen activator (TPA)) streptokinase; urokinase: anisoylated plasminogen-streptokinase activator complex; pro-urokinase (pro-UK); rTPA (alteplase or activase); rPro-UK; abbokinase; eminase; sreptase anagrelide hydrochloride; bivalirudin; dalteparin sodium; danaparoid sodium; dazoxiben hydrochloride; efegatran sulfate; enoxaparin sodium; ifetroban; ifetroban sodium; tinzaparin sodium; retaplase; trifenagrel; warfarin; and dextrans.

Anti-platelet agents include clopridogrel; sulfinpyrazone; aspirin; dipyridamole; clofibrate; pyridinol carbamate; PGE; glucagon; antiserotonin drugs; caffeine; theophyllin; pentoxifyllin; ticlopidine; and anagrelide.

Lipid reducing agents include gemfibrozil, cholystyramine, colestipol, nicotinic acid, probucol, lovastatin, fluvastatin, simvastatin, atorvastatin, pravastatin, and cirivastatin.

Direct thrombin inhibitors include hirudin, hirugen, hirulog, agatroban, PPACK, and thrombin aptamers.

Glycoprotein IIb/IIIa receptor inhibitors include both antibodies and non-antibodies, and include but are not limited to ReoPro (abcixamab), lamifiban, and tirofiban.

Calcium channel blockers are a chemically diverse class of compounds having important therapeutic value in the control of a variety of diseases including several cardiovascular disorders, such as hypertension, angina, and cardiac arrhythmias (Fleckenstein, Cir. Res. (1983) 52:13-16; Fleckenstein, Experimental Facts and Therapeutic Prospects, John Wiley, New York (1983); McCall, D., Curr. Pract. Cardiol. (1985) 10:1-11). Calcium channel blockers are a heterogenous group of drugs that prevent or slow the entry of calcium into cells by regulating cellular calcium channels. (Remington, The Science and Practice of Pharmacy, Nineteenth Edition, Mack Publishing Company, Eaton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, aminone, amlodipine, bencyclane, felodipine, fendiline, flunarizine, isradipine, nicardipine, nimodipine, perhexylene, gallopamil, tiapamil and tiapamil analogues (such as 1993RO-11-2933), phenyloin, barbiturates, and the peptides dynorphin, omega-conotoxin, and omega-agatoxin, and pharmaceutically acceptable salts thereof.

Beta-adrenergic receptor blocking agents are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, befunolol, betaxolol, bunitrolol, carteolol, celiprolol, hydroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hyd-roxypropoxy)-3-pyridenecarbonitril HCl, 1-butylamino-3-(2,5-dichlorophenoxy-)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, -7-(2-hydroxy-3-t-butylaminpropoxy)phthalide. These compounds can be used as isomeric mixtures, or in their respective levorotatory or dextrorotatory forms.

Cyclooxygenase-2 (COX-2) is an enzyme complex present in most tissues that produces various prostaglandins and thromboxanes from arachidonic acid. A number of selective COX-2 inhibitors are known in the art. These include, but are not limited to, those described in U.S. Pat. Nos. 5,474,995, 5,521,213, 5,536,752, 5,550,142, 5,552,422, 5,604,253, 5,604,260, 5,639,780, 5,677,318, 5,691,374, 5,698,584, 5,710,140, 5,733,909, 5,789,413, 5,817,700, 5,849,943, 5,861,419, 5,922,742, 5,925,631, and 5,643,933. A number of the above-identified COX-2 inhibitors are prodrugs of selective COX-2 inhibitors and exert their action by conversion in vivo to the active and selective COX-2 inhibitors. The active and selective COX-2 inhibitors formed from the above-identified COX-2 inhibitor prodrugs are described in detail in PCT/WO95/00501, PCT/WO95/18799, and U.S. Pat. No. 5,474,995. Given the teachings of U.S. Pat. No. 5,543,297, a person of ordinary skill in the art would be able to determine whether an agent is a selective COX-2 inhibitor or a precursor of a COX-2 inhibitor.

Angiotensin system inhibitors are capable of interfering with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na⁺ in plasma tend to activate the system, while factors that increase these parameters tend to suppress its function.

Angiotensin I and angiotensin II are synthesized by the enzymatic renin-angiotensin pathway. The synthetic process is initiated when the enzyme renin acts on angiotensinogen, pseudoglobulin in blood plasma, to produce the decapeptide angiotensin I. Angiotensin I is converted by angiotensin converting enzyme (ACE) to angiotensin II (angiotensin-[1-8]octapeptide). The latter is an active pressor substance which has been implicated as a causative agent in several forms of hypertension in various mammalian species, e.g., humans.

Angiotensin (renin-angiotensin) system inhibitors are compounds that act to interfere with the production of angiotensin II from angiotensinogen or angiotensin I or interfere with the activity of angiotensin II. Such inhibitors are well known to those of ordinary skill in the art and include compounds that act to inhibit the enzymes involved in the ultimate production of angiotensin II, including renin and ACE. They also include compounds that interfere with the activity of angiotensin II, once produced. Examples of classes of such compounds include antibodies (e.g., to renin), amino acids and analogs thereof (including those conjugated to larger molecules), peptides (including peptide analogs of angiotensin and angiotensin I), pro-renin related analogs, etc. Among the most potent and useful renin-angiotensin system inhibitors are renin inhibitors, ACE inhibitors, and angiotensin II antagonists. In a preferred embodiment of the invention, the renin-angiotensin system inhibitors are renin inhibitors, ACE inhibitors, and angiotensin II antagonists.

Angiotensin II antagonists are compounds which interfere with the activity of angiotensin II by binding to angiotensin II receptors and interfering with its activity. Angiotensin II antagonists are well known and include peptide compounds and non-peptide compounds. Most angiotensin II antagonists are slightly modified congeners in which agonist activity is attenuated by replacement of phenylalanine in position 8 with some other amino acid; stability can be enhanced by other replacements that slow degeneration in vivo. Examples of angiotensin II antagonists include: peptidic compounds (e.g., saralasin, [(San¹)(Val⁵)(Ala⁸)]angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4, 5,6,7-tetrahydro-1H-imidazo[4,5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenyl and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(-1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF 108566 (E-alpha-2-[2-butyl-1-(carboxy phenyl)methyl]1H-imidazole-5-yl[methylane]-2-thiophenepropanoic acid, Smith Kline Beecham Pharmaceuticals, PA); Losartan (DUP753/MK954, DuPont Merck Pharmaceutical Company); Remikirin (RO42-5892, F. Hoffman LaRoche AG); A₂ agonists (Marion Merrill Dow) and certain non-peptide heterocycles (G.D. Searle and Company).

Angiotensin converting enzyme (ACE) is an enzyme which catalyzes the conversion of angiotensin Ito angiotensin II. ACE inhibitors include amino acids and derivatives thereof, peptides, including di and tri peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Renin inhibitors are compounds which interfere with the activity of renin. Renin inhibitors include amino acids and derivatives thereof, peptides and derivatives thereof, and antibodies to renin. Examples of renin inhibitors that are the subject of United States patents are as follows: urea derivatives of peptides (U.S. Pat. No. 5,116,835); amino acids connected by nonpeptide bonds (U.S. Pat. No. 5,114,937); di and tri peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

Agents that bind to cellular adhesion molecules and inhibit the ability of white blood cells to attach to such molecules include polypeptide agents. Such polypeptides include polyclonal and monoclonal antibodies, prepared according to conventional methodology. Such antibodies already are known in the art and include anti-ICAM 1 antibodies as well as other such antibodies. Significantly, as is well-known in the art, only a small portion of an antibody molecule, the paratrope, is involved in the binding of the antibody to its epitope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd Fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (Frs), which maintain the tertiary structure of the paratope (see, in general, Clar, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or Fr and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences. The present invention also includes so-called single chain antibodies.

Thus, the invention involves polypeptides of numerous size and type that bind specifically to cellular adhesion molecules. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using, e.g., m13, fd, or lambda phage), displaying inserts from 4 to about 80 amino acid residues using conventional procedures. The inserts may represent, for example, a completely degenerate or biased array. One then can select phage-bearing inserts which bind to the cellular adhesion molecule. This process can be repeated through several cycles of reselection of phage that bind to the cellular adhesion molecule. Repeated rounds lead to enrichment of phage bearing particular sequences. DNA sequences analysis can be conducted to identify the sequences of the expressed polypeptides. The minimal linear portion of the sequence that binds to the cellular adhesion molecule can be determined. One can repeat the procedure using a biased library containing inserts containing part of, or all of, the minimal linear portion plus one or more additional degenerate residues upstream or downstream thereof. Yeast two-hybrid screening methods also may be used to identify polypeptides that bind to the cellular adhesion molecules. Thus, cellular adhesion molecules, or a fragment thereof, can be used to screen peptide libraries, including phage display libraries, to identify and select peptide binding partners of the cellular adhesion molecules.

### Preventative Therapy

In a patient diagnosed with any of the conditions of Table 1, e.g., heart disease (e.g., coronary heart disease or ischemic heart injury) or degenerative disease (e.g., a neurodegenerative disease, such as Alzheimer's disease or Huntington's disease), any of the above therapies may be administered before the occurrence of the disease phenotype. In particular, compounds shown to decrease necrosis may be administered by any standard dosage and route of administration (as described above).

The methods of the instant invention may be used to decrease necrosis of a cell or to treat disorders described herein in any subject, for example, humans; domestic pets, such as, for example, canines or felines; or livestock.

Chemical Compounds that Decrease Cell Necrosis

After a cell receives an initial assault, one or both of the apoptosis or necrosis mechanisms of cell death may be activated. Several chemical assaults can be used to induce cell death, including exposure to tumor necrosis factor alpha (TNFα) and β-amyloid protein. Various cell types can also be used, including human neuroblastoma cells (SH-SY5Y) and human Jurkat T cells. In order to block the apoptosis mechanism, a general caspase inhibitor, Cbz-valine-alanine-aspartyl fluoromethyl ketone (zVAD-fink, Polyerino and Patterson, J. Biol. Chem. 272:7013-7021, 1997), can be given. This compound inhibits all caspases and consequently disrupts the apoptosis pathway. Any resulting cell death can be assumed to arise from the necrosis mechanism. After administration of zVAD-fmk and TNFα to the cells, test compounds can be applied to the cells in an attempt to rescue them. The compounds found to restore cell viability using this protocol are considered to be inhibitors of the necrosis pathway.

For example, in one approach, zVAD-fmk may be added to the culture media of cells at high density (for example, 5×10⁵ or 7.5×10⁵ cells/ml), which are capable of undergoing necrosis in response to zVAD-fmk/TNFα. Candidate molecules, for example, chemical compounds from a chemical library, such as, for example, the library of compounds from ChemBridge Research Laboratories, San Diego, Calif.) are added, in varying concentrations to the cells, and the cells are then exposed to TNFα.

The occurrence of necrosis of the treated cells is then measured, for example, by measuring the cellular ATP level of the cells exposed to zVAD-fmk/TNFα (Crouch et al., J. Immunol. Methods (1993) 160:81-88; Storer et al., Mutat. Res. (1996) 368:59-101; and Cree et al., Toxicol. In Vitro (1997) 11:553-556). The level of necrosis in the presence of the candidate molecule is compared to the level of necrosis in the absence of the candidate molecule, all other factors (e.g., cell type and culture conditions) being equal. The importance of zVAD-fmk in the invention is to block cell death that may occur by apoptosis, so that cell death by necrosis can be fully unmasked.

In a second approach, a cell may be exposed to a candidate molecule that decreases necrosis at the same time it is exposed to either zVAD-fmk or TNFα. In a third approach, a cell may be exposed to zVAD-fmk and TNFα first, and then to a candidate compound. The level of necrosis that occurs following each of these approaches is measured as described above.

The effect of candidate molecules on necrosis induced by cell death stimuli, for example, TNFα or DMSO, may also be measured by other methods, for example, vital dye staining, using dyes such as trypan blue or acridine orange/ethidium bromide.

Compounds that decrease necrosis may be purified or substantially purified, or may be one component of a mixture of compounds, such as a pool of chemical compounds. In an assay of a mixture of compounds, the occurrence of necrosis is tested against progressively smaller subsets of the compound pool (e.g., produced by standard purification techniques such as HPLC or FPLC) until a single compound or minimal number of effective compounds is demonstrated to decrease necrosis. A molecule that promotes a decrease in necrosis induced by zVAD-fmk/TNFα is considered particularly useful in the invention; such a molecule may be used, for example, as a therapeutic to decrease necrosis, in a patient with a condition in which necrosis occurs, such as a neurodegenerative disease.

Chemical compounds that are found, by the methods described above, to effectively decrease necrosis induced, for example, by zVAD-fmk/TNFα in an in vitro system may be tested further in animal models. Particularly useful animal models include mouse and rat models of cell death, ischemic brain or heart injury or other ischemic injuries, head trauma, neurodegenerative diseases, coronary heart disease, and septic shock. Examples of such models include SOD or Huntington's disease gene transgenic mice, and other known models, such as those described by Li et al., Hum. Mol. Genet. (1999) 8:1227-12236; Levine et al., Neurosci. Res. (1999) 58:515-532; Vukosavic et al., J. Neurochem. (1999) 73:2460-2468; Gruney, J. Neurol. Sci. 152 suppl. (1997) 1:S67-73; Deshmukh et al., Am. J. Physiol. (1997) 273 (4 Pt 1):C1130-1135; and Isibashi et al., J. Immunol. (1999) 163:5666-5677. Compounds which demonstrate an ability to decrease necrosis in in vivo models may be used as therapeutics to prevent necrosis, as appropriate.

Identification of Chemical Compounds that Decrease zVAD-fmk/DMSO-Induced Cell Necrosis

Methods for the identification of chemical compounds that decrease cell necrosis induced, for example, by zVAD-fmk/DMSO at a low cell density (e.g., 1×10⁵ cells/ml) are achieved essentially as described herein, except, the inducer of necrosis is zVAD-fmk/DMSO, rather than zVAD-fink/TNFα.

### Alternate Screening Assays

Any method for measuring protein interactions or inhibition of the activity of a target molecule may be utilized. Such methods include, but are not limited to fluorescence polarization assays, mass spectrometry (Nelson and Krone, J. Mol. Recognit. (1999) 12:77-93), surface plasmon resonance (Spiga et al., FEBS Lett. (2002) 511:33-35; Rich and Mizka, J. Mol. Recognit. (2001) 14:223-228; Abrantes et al., Anal. Chem. (2001) 73:2828-2835), fluorescence resonance energy transfer (FRET) (Bader et al., J. Biomol. Screen (2001) 6:255-264; Song et al., Anal. Biochem (2001) 291:133-41; Brockhoff et al., Cytometry (2001) 44:338-248), bioluminescence resonance energy transfer (BRET) (Angers et al., Proc. Natl. Acad. Sci. USA (2000) 97:3684-3689; Xu et al., Proc. Natl. Acad. Sci. USA (1999) 96:151-156), fluorescence quenching (Engelborghs, Spectrochim. Acta A. Mol. Biomol. Spectrosc. (1999) 57:2255-2270; Geoghegan et al., Bioconjug. Chem. (2000) 11:71-77), fluorescence activated cell scanning/sorting (Barth et al., J. Mol. Biol. (2000) 301:751-757), ELISA, and radioimmunoassay (RIA).

### Candidate Compounds

In general, candidate compounds used in the screening assays of the invention are identified from large libraries of both natural products, synthetic (or semi-synthetic) extracts or chemical libraries, according to methods known in the art.

### Cell Viability Assays

Cell viability assays which can assess compounds of the present invention include the following.

U937 cells can be plated in 384-well plates at 5,000-10,000 cells per well in 40-μl phenol red-free RPMI 1640 medium containing 100 μM zVAD.fmk and 40 ng ml⁻¹ human TNFα using a Multidrop dispenser (Thermo Electron), followed by addition of 100 nl of the DiverSetE (5 mg ml⁻¹ in DMSO, Chembridge) using a Seiko-based custom-built pin transfer robot (Institute of Chemistry and Cell Biology, Harvard Medical School). After 72 hours, cell viability can be assessed using a luminescence-based ATP assay (ATPLite-M, PerkinElmer). Cells not treated with TNFα may be dispensed in each plate as a positive control.

Cells can be seeded in 96-well plates (white plates for luminescent assays; black plates for fluorescent assays; clear plates for MTT assay) at the density of 5,000-10,000 cells per well for adherent cells or 20,000-50,000 cells per well for suspension cells in 100 μl of the appropriate phenol red-free media. After incubation, one may determine cell viability using one of the following methods.

For the ATP assay, luminescence-based commercial kits (CellTiter-Glo, Promega or ATPLite-M, PerkinElmer) can be used, and the luminescence analyzed using a Wallac Victor II plate reader (PerkinElmer).

For the Sytox assay, cells can be incubated with 1 μM Sytox Green reagent for thirty minutes at 37° C., and then a fluorescent reading can be performed. Addition of 411 of 20% Triton X-100 solution into each well produces maximal lysis, and the cells should be incubated for one hour at 37° C., and then second reading should be performed. The ratio of values can be calculated (percentage of dead cells in each well) before and after Triton treatment and normalized to the relevant controls not subjected to cytotoxic stimuli.

For the MTT assay, the CellTiter 96 AQᵤₑₒᵤₛ. Non-Radioactive Cell Proliferation Assay kit (Promega) can be used. For PI exclusion assays, one can add 2 μg ml⁻¹ PI into the medium and immediately analyze samples using FACSCalibur (BD Biosciences).

For the PI-annexin V assay, one can use the ApoAlert Annexin V-EGFP Apoptosis Kit (Clontech). For DioC6 staining, one can incubate cells with 40 nM DiOC₆ for thirty minutes at 37° C., then wash once and analyze in FACSCalibur.

For ROS analysis, one can incubate cells with 5 μM dihydroethidium (Molecular Probes) for thirty minutes at 37° C., then wash once and analyze in FACSCalibur. One may acquire bright-field images of the cells using an Axiovert 200 microscope (Zeiss).

### Transient Focal Cerebral Ischemia in the Mouse

A mouse model which may be used to analyze compounds of the present invention is as follows: One can anesthetize spontaneously breathing adult male SV-129 mice (19-23 g; Taconic Farms) with 2% isoflurane and maintain them on 0.8-1% isoflurane in 70% N₂O and 30% O₂ using a Fluotec 3 vaporizer (Colonial Medical). One may then occlude the left MCA with an intraluminal 8-0 nylon monofilament (Ethicon) coated with a mixture of silicone resin (Xantopren, Bayer Dental) and a hardener (Elastomer Activator, Bayer Dental). Once the procedure is complete (which may last approximately fifteen minutes), the anesthesia can be discontinued. One may briefly reanesthetize animals two hours later with isoflurane, and then withdraw the filament. Eighteen hours after reperfusion one may divide forebrains into five coronal (2-mm) sections using a mouse brain matrix (RBM-2000C; Activational Systems), and stain the sections with 2% 2,3,5-triphenyltetrazolium chloride (Sigma). One may quantify the infarct areas using an image-analysis system (Bioquant IV, R & M Biometrics) and calculate infarct volume directly by adding the infarct volume in each section.

For drug administration, one may dissolved Nec-5 or other derivatives (such as 4% methyl-β-cyclodextrin (Sigma) solution in PBS) and administered it by intracerebroventricular administration. For preocclusion delivery, one may perform injections five minutes before the onset of 2-h MCAO occlusion and immediately after the cessation of the occlusion, at the time of the reperfusion. For postocclusion delivery, one may performed injections at the time of reperfusion after two hours of MCAO as well as two hours after the onset of reperfusion. In the case of infusion, one may infuse (for example, 20 μl) of compound over a thirty minute time period. In the case of injection six hours after occlusion, one may injected a single (for example, 4-μl) dose. In the case of zVAD.fmk administration, one may add it to the Nec-5 formulation and administer it to the animal.

One can prepare mouse embryonic fibroblasts as in Nakagawa et al., Nature (2000) 403:98-103, and immortalize through infection with SV-40-encoding retrovirus. Atg5^{-/-} MEF cells have been previously described (see, e.g., Kuma et al., Nature (2004) 432:1032-1036).

### Immunofluorescence

One can analyze the compounds of the present invention by immunofluorescence as follows: One may wash Balbc 3T3 cells in PBS, fix the cells in 4% formaldehyde for fifteen minutes at 25° C., rinse them twice in PBS, and permeabilize/block in 0.4% Triton X-100, 10% normal goat or donkey serum (Jackson Immunoresearch) in PBS for 30 minutes at 25° C. The samples can be incubated with the appropriate primary antibodies, diluted according to the manufacturer's instructions in 0.1% Triton, 1% serum in PBS, for sixteen hours at 4° C., followed by three washes with PBS and incubated with fluorophore-conjugated secondary antibodies diluted 1:200 in the same buffer as primary antibodies for thirty minutes at 25° C. After two washes with PBS, the cells may be stained with TO-PRO-3 or phalloidin-TRITC, diluted in PBS according to manufacturer's instructions, for ten minutes at 25° C., washed with PBS, and mounted using ProLong Antifade kit (Molecular Probes). Images can be acquired using a Nikon spinning disk confocal microscope and analysis of these images can be conducted using Metamorph software (Universal Imaging).

### Propidium Iodide DNA Content Analysis

One can analyze compounds of the present invention for DNA content as follows: After the appropriate treatment, Jurkat cells can be washed once, resuspended in PBS, and then fixed by adding four volumes of ice cold 100% ethanol. These cells should remain on ice for approximately one hour, after which the fixing solution may be discarded, the cells washed once in PBS, resuspended in PBS supplemented with 50 μg/ml PI and 5 μg/ml RNAse A (Sigma), and incubated in the dark for fifteen minutes at 37° C. followed by analysis in FACSCalibur. The data can be analyzed using ModFit software (Verity Software House).

### Immunoblotting

One can analyze compounds of the present invention by immunoblotting as follows: The cells can be lysed in 20 mM HEPES, pH 7.5, 150 mM NaCl, 1% Triton X-100, 10 mM tetrasodium pyrophosphate, 100 mM NaF, 17.5 mM β-glycerophosphate buffer supplemented with Complete Mini Protease Inhibitor tablet (Roche). One can determine of the protein concentrations using Bio-Rad Protein Assay reagent and subjecting equal amounts of protein to Western blotting using antibodies. In case of ischemic brain samples, one can dissect out injured regions of the cortexes, lyse them in RIPA buffer (50 mM Tris-HCl, pH 8.0, 150 mM NaCl, 5 mM EDTA, 0.1% SDS, 0.5% sodium deoxycholate, 1% NP-40, supplemented with Complete Mini protease inhibitors) and subject equal amounts of proteins to western blotting. Results of western blotting can be quantified using Scion Image software (Scion Corporation).

### Examples 1 to 7

We have screened a chemical library of approximately 100,000 compounds for chemical inhibitors of the necrotic death of human monocytic U937 cells induced by TNFα and zVAD-fmk, which was used as an operational definition of necroptosis (Degterev et al., Nature Chem. Biol. (2005) 2:112-119; Teng et al., Bio. Med. Chem. Lett. (2005) 15:5039-5044). This screen resulted in the selection of several necroptosis inhibitors which efficiently blocked necroptotic death (Li and Beg, J. Virol. (2000) 74:7470-7477; Lin et al., J. Biol. Chem. (2004) 279:10822-10828; Wilson et al., Cell Death Differ. (2002) 9:1321-1333). Here we describe the novel necrostatin, Nec-5, depicted below. Although Nec-5 was selected in a screen in the presence of zVAD-fmk, its action is not dependent upon pharmacological inhibition of caspases. This finding is consistent with the direct activation of necroptosis when induction of apoptosis is abolished by genetic inactivation of apoptotic machinery (Lo et al., Nat. Rev. Neurosci. (2003) 224: 29-55; Gwag et al., Neuroscience (1995) 68:615-619; Rosenbaum et al., J. Neurosci. Res. (2000) 61:686-692; Martin-Villalba et al., J. Neurosci. (1999) 19:3809-3817; Martin-Villalba et al., Cell Death Differ. (2001) 8:679-686.

Nec-5 prevents the death of TNFα treated FADD-deficient Jurkat cells, which are unable to active caspases in response to DR signaling, even in the absence of zVAD-fmk (Chan, J. Biol. Chem. (2003) 278:51613-51621). Because the induction of necroptosis in FADD-deficient Jukart cells does not rely on the presence of other chemicals, e.g. zVAD-fmk, this system was used to determine that the effective concentration for half-maximum response (EC₅₀) for Nec-5 was 0.24 μM. Herein we describe the structure activity analysis of Nec-5 analogs.

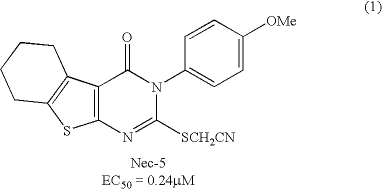

Chemically, Nec-5 is known as 3-p-methoxyphenyl-5,6-tetramethylenothieno[2,3-d]pyrimidin-4-one-2-mercapto ethylcyanide; however, its method of synthesis has not been reported. Our synthetic protocol is as follows: On reacting compound (3) with p-methoxyphenyl isothiocyanate, a thiourea analog (4) is generated (Scheme 2). Cyclization of the latter in ethanolic HCl provides 2-mercapto-3-p-methoxyphenyl-5,6-tetramethylenothieno[2,3-d]pyrimidin-4-one (5) (Gewald et al., Chem. Ber. (1966) 99:94-100; Tranberg et al., J. Med. Chem. (2002) 45:382-389; Gutschow et al., J. Med. Chem. (1999) 42:5437-5447; Sabnis, Sulfur Rep. (1994) 16:1-17; Sabnis and Rangnekar, J. Heterocyclic. Chem. (1999) 36:333-345; Vishnu et al., J. Heterocyclic. Chem. (1981) 18:1277; Devani et al., J. Pharm. Sci. (1976) 65:660-664; Leistner et al., Synthesis (1987) 466-470; Modica et al., Bioorg & Med. Chem. Lett. (2000) 10:1089; Duval et al., Bioorg & Med. Chem. Lett. (2005) 15:1885-1890), which gave Nec-5 (1) in 92% yield upon reaction with BrCH₂CN in the presence of potassium hydroxide (Vittenet, Bull. Soc. Chim. (1899) 21:955).

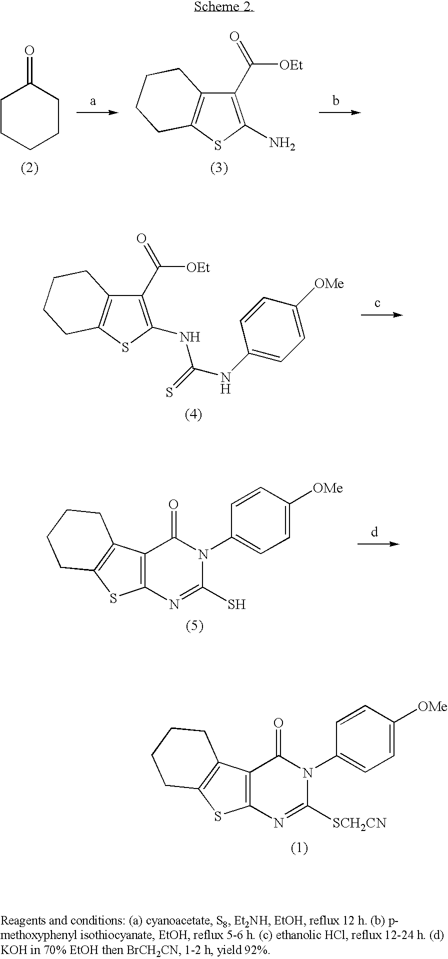

### Example 1

### Influence of Substituent on the Sulfur Atom of Nec-5

For the study of the influence of substituents of sulfur atom of Nec-5 on their bioactivities, a series of compounds of Formula (V) were prepared by reaction of compound (5) with RX in the presence of potassium hydroxide.

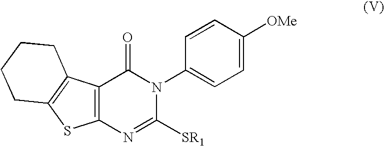

TABLE 2
Structure and activity of compounds of Formula (V)

| | | Yield | EC₅₀ | Max |
|---|---|---|---|---|
| Compound | R₁ | (%)^{a)} | (μM)^{b)} | Prot (%)^{c)} |
| 1 | —CH₂CN | 92 | 0.24 | 100 |
| 6 | Me | 87 | 0.24 | 71 |
| 7 | Et | 78 | inactive | — |
| 8 | n-Pr | 54 | inactive | — |
| 9 | n-Bu | 87 | inactive | — |
| 10 | Pent | 77 | inactive | — |
| 11 | Hex | 84 | inactive | — |
| 12 | —CH₂CH═CH₂ | 88 | inactive | — |
| 13 | —CH₂C≡CH | 80 | 6.08 | 80.7 |
| 14 | —CH₂C₆H₅ | 92 | inactive | — |
| 15 | —CH₂(C₆H₄Me-4) | 88 | inactive | — |
| 16 | —CH₂(C₆H₄OMe-4) | 92 | inactive | — |
| 17 | —CH₂(C₆H₄NO₂-4) | 85 | inactive | — |
| 18 | —CH₂COMe | 80 | inactive | — |
| 19 | —CH₂COOMe | 79 | inactive | — |
| 20 | —CH₂CONH₂ | 67 | inactive | — |
| 21 | —COMe | 91 | inactive | — |
| 22 | —COC₃H₇-n | 87 | inactive | — |
| 23 | —COC₆H₅ | 92 | inactive | — |
| 24 | —CH₂CH₂CN | 65 | 5.28 | 70 |
| 25 | —CH₂Cl | 45 | 2.22 | 85 |
| 26 | —CH₂NO₂ | 36 | inactive | — |
| 27 | —CH₂C(O)NH(C₆H₄CF₃- | 76 | inactive | — |
| | 2) | | | |
| 28 | —CH₂CH(OH)CH₃ | 68 | inactive | — |
| 29 | —CH₂COOH | 65 | inactive | — |
| 30 | Me (Sulfoxide) | — | inactive | — |
| 31 | Me(Sulfone) | 87 | inactive | — |

^{a)}Yield % denotes percentage yield in the final reaction of synthesis;
^{b)}EC₅₀ is the effective concentration for half-maximum response;
^{c)}Max Protection represents max viability obtained in the presence of a compound.

As shown in Table 2, of all of the compounds tested, only few types of changes retained activity in the necroptosis assay based on the treatment of FADD-deficient Jurkat cells with TNFα described herein. It should be noted that while some of the compounds afforded complete 100% protection from necroptosis restoring viability to control, a number of modifications resulted in not only a change in EC₅₀ values, but also a decrease in the degree of protection, as determined by non-linear regression analysis of the viability data using GraphPad Prizm scientific statistical software package.

Experimental data in Table 2 shows that substitution of ethylcyanide moiety by a methyl group in Nec-5 (compound (6) in Table 2) reserves its activity to a significant extent. On the other hand, further extension of carbon chain on sulfur (compounds (7) to (11)) resulted in the loss of activity. Compound (24), in which methyl cyanide side chain of Nec-5 (i.e., compound (1)) was replaced with —CH₂CH₂CN, retained some activity. To a lesser extent, compounds (13) and (25) exhibited some activity, while introduction of electron withdrawing group (EWG), for instance, compounds (14), (19), and (26), completely destroyed the activity of the molecule. Overall, this data suggests that the R₁ position of Nec-5 affords some limited flexibility and can tolerate, for example, an ethylcyanide side chain or S-methylmoiety, and that the presence of a thioether bond is greatly preferred. Oxidation of the methylthiogroup to the corresponding sulfoxide (i.e., compound (30)) or sulfone (i.e., compound (31)) lead to a complete loss of activity.

### Example 2

### Influence of N-Substituents of Pyrimidinone Part of Nec-5

For the study of the influence of the aryl substituents, 3-aryl-5,6-tetramethylen-othieno[2,3-d]pyrimidin-4-one-2-mercapto ethylcyanide compounds of Formula (VI), in which R₅ was introduced into benzene ring, were prepared. Since introduction of the methylmercapto moiety resulted in substantial activity (as previously described), 2-methylthio-3-aryl-5,6-tetramethylenothieno[2,3-d]pyrimidin-4-one compounds of Formula (VII) were also prepared.

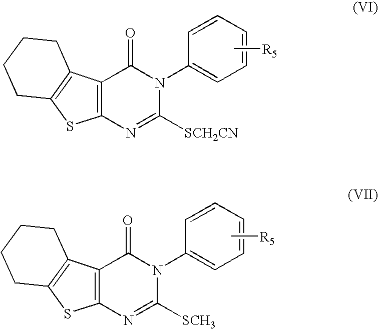

To prepare compounds of Formula (VI), compound (1) was reacted with aryl isothiocyanate derivatives. The resulting thiourea analog was smoothly cyclized in ethanolic HCl to form 2-mercapto-3-aryl-5,6-tetramethylenothieno[2,3-d]pyrimidin-4-ones. The latter was reacted with BrCH₂CN in the presence of potassium hydroxide to give compounds as listed

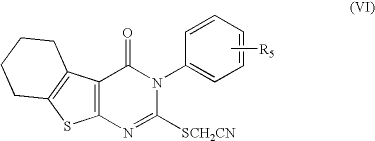

TABLE 3
Structure and activity of compounds of Formula (VI)

| Compound | R₅ | Yield (%) | EC₅₀ (μM) | Max Prot (%) |
|---|---|---|---|---|
| 32 | H | 87 | inactive | — |
| 33 | 2-OMe | 58 | 17.8 | 51.2 |
| 34 | 3-OMe | 87 | 1.46 | 87.7 |
| 1 | 4-OMe | 92 | 0.24 | 100 |
| 35 | 4-OEt | 85 | 0.55 | 83.8 |
| 36 | 4-OBn | 83 | inactive | — |
| 37 | 2-Me | 87 | inactive | — |
| 38 | 4-Me | 80 | 1.80 | 48.6 |
| 39 | 4-F | 81 | 0.24 | 85.1 |
| 40 | 3-Cl | 85 | 5.10 | 66.9 |
| 41 | 4-Cl | 90 | 8.50 | 55.0 |
| 42 | 4-Br | 87 | inactive | — |
| 43 | 3,4-Me₂ | 91 | 16.7 | 45.0 |
| 44 | 3,4-Cl₂ | 76 | 5.70 | 39.0 |
| 45 | 3,4-F₂ | 74 | inactive | — |
| 46 | 2,4-(OMe)₂ | 47 | inactive | — |
| 47 | 3,4-(OMe)₂ | 52 | 5.70 | 57.0 |
| 48 | 3,4-O₂(CH₂) | 79 | 0.89 | 100 |
| 49 | 4-SMe | 55 | 2.33 | 70.0 |
| 50 | 2-Me, 4-Cl | 74 | inactive | — |
| 51 | 4-OCF₃ | 81 | inactive | — |

As shown in Table 3, compound (32), with an unsubstituted phenyl ring, was inactive, while introduction of a para-methyl group to the benzene ring (i.e., compound (38)) or replacement of a methoxy group with ethoxy group (i.e., compound (35)) retained some activity. An increase in the R₅ substituent size, for example, as observed with compound (46), wherein R₅ is para-benzyloxy (—OBn), eliminated activity, indicating the important role of the R₅ para-methoxy substituent in Nec-5, and further indicating that increasing the steric bulk of R₅ is not favorable to activity. Interestingly, the R₅ substituent para-OCF₃ resulted in a loss of activity (compound (51)), while the R₅ substituent para-F, for example, as observed with compound (39), retained significant activity. Moreover, the activity significantly decreased when the para-F substituent (compound (39)) was replaced by a para-Cl substituent (compound (41)) or a para-Br substituent (compound (42)). Compound (48) (wherein R₅=3,4-O₂(CH₂)) showed good activity, indicating that the target's highly restricted binding pocket shows a strong preference for the methoxy group.

Similar to the synthetic method described above, compounds of Formula (VII) were prepared by reacting compound (1) with aryl isothiocyanate derivatives. The resulting thiourea analog was smoothly cyclized in ethanolic HCl to form 2-mercapto-3-aryl-5,6-tetramethylenothieno[2,3-d]pyrimidin-4-one derivatives. The later was reacted with MeI in the presence of potassium hydroxide to give compounds as listed in Table 4.

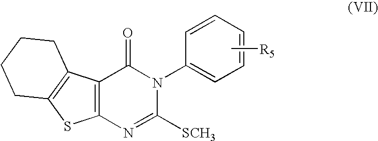

TABLE 4
Structure and activity of compounds of Formula (VII)

| Compound | R₅ | Yield (%) | EC₅₀ (μM) | Max Prot (%) |
|---|---|---|---|---|
| 6 | 4-OCH₃ | 87 | 0.24 | 71 |
| 52 | 4-OBn | 75 | inactive | — |
| 53 | 4-F | 64 | 1.57 | 61.2 |
| 54 | 4-Br | 77 | inactive | — |
| 55 | 3,4-Me₂ | 82 | 16.7 | 38.0 |
| 56 | 3,4-Cl₂ | 71 | inactive | — |
| 57 | 2,4-(OMe)₂ | 58 | inactive | — |
| 58 | 3,4-(OMe)₂ | 69 | 16.7 | 54.5 |
| 59 | 3,4-O₂(CH₂) | 75 | inactive | — |
| 60 | 3-SMe | 67 | inactive | — |
| 61 | 2-Me, 4-Cl | 71 | inactive | — |
| 62 | 4-OCF₃ | 84 | inactive | — |

As shown in Table 4, while compounds (53) and (58) showed some activity, albeit significantly lower than observed with Formula (VI) analogs (compound (39) and compound (47)), all of other derivatives were inactive. Comparison of the data of Table 3 with the data of Table 4 indicates that the R₁ ethylcyanide moiety is greatly preferred over R₁ methylthio moiety.

### Example 3

### Influence of Substituents on Thiophene Ring of Nec-5

In order to study the influence of substituents on thiophene ring of Nec-5,3-p-methoxyphenyl-5,6-disubstituted thieno[2,3-d]pyrimidin-4-one-2-mercapto ethylcyanide compounds (corresponding to compounds of Formula (XII)) were synthesized, in which the fused cyclohexyl ring of Nec-5 was replaced by substituents R₃ and R₄. 2-methylthio-3-p-methoxyphenyl-5,6-disubstituted thieno[2,3-d]pyrimidin-4-one compounds (corresponding to compounds of Formula (XIII)) were also synthesized.

Compounds of Formula (XII) series were generated from the corresponding 2-amino-3-carbethoxythiophenes (IX), which reacts with p-methoxyphenylisothiocyanate to obtain thiourea analogs (X), and then cyclized in ethanolic HCl solution to form 2-mercapto[2,3-d]pyrimidin-4-ones (XI) (Scheme 3). The latter gave compounds of Formula (XII) upon reaction with BrCH₂CN in the presence of potassium hydroxide.

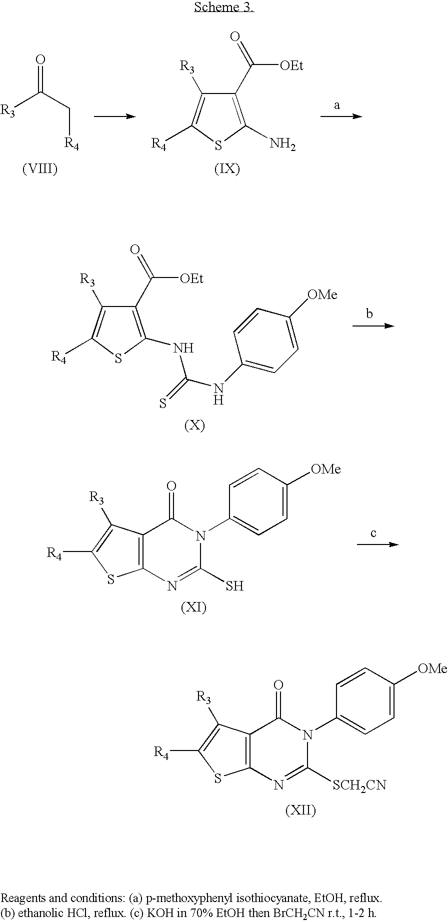

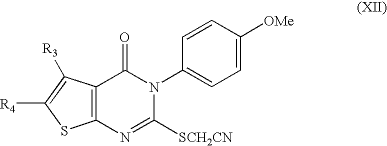

TABLE 5
Structure and activity of compounds of Formula (XII)

| | | | | EC₅₀ | Max |
|---|---|---|---|---|---|
| Compound | R₃ | R₄ | Yield(%) | (μM) | Prot(%) |
| 63 | H | H | 80 | inactive | — |
| 64 | H | Me | 65 | inactive | — |
| 65 | H | Et | 70 | inactive | — |
| 66 | Me | H | 88 | inactive | — |
| 67 | Me | Me | 91 | 0.45 | 100 |
| 68 | Me | Et | 89 | 5.26 | 86.8 |
| 69 | Me | n-Pr | 72 | inactive | — |
| 70 | Me | i-Pr | 75 | inactive | — |
| 71 | Me | C₁₄H₂₉ | 71 | inactive | — |
| 72 | Et | Me | 77 | 1.08 | 100 |

| | | | | |
|---|---|---|---|---|
| 73 | —(CH₂)₃— | 81 | 0.45 | 100 |
| 1 | —(CH₂)₄— | 92 | 0.24 | 100 |
| 74 | —(CH₂)₅— | 65 | 0.96 | 83 |
| 75 | —CH₂CH₂CHCH₃CH₂— | 72 | 150 | 40 |
| 76 | —CH═CHCH═CH— | 44 | 0.18 | 83.3 |
| 77 | —CH₂CH₂NEtCH₂— | 37 | inactive | — |
| 78 | —CH₂CH₂N(i-Pr)CH₂— | 54 | inactive | — |

As shown in Table 5, compounds (63) to (66), which contain hydrogen in the R₃ and/or R₄ position of 5,6-thiophene ring, were completely inactive. When R₃ and R₄ were both methyl groups (compound (67)), a high degree of activity is retained. Limited extension of R₃ preserved activity to a significant extent (compound (72)), while extending the R₄ position was significantly more detrimental. For example, compound (68) (R₄=Et) displayed EC₅₀ of 5.26 μM and 86.8% protection, and compounds (69) and (70) were found inactive. Thus, the experimental data demonstrates that while R₃ and R₄ contribute to compound activity, extension of the hydrocarbon chain beyond methyl at the R₄ position and, to lesser extent, at the R₃ position, is detrimental for activity.

Changing the size of the aliphatic 6-membered ring of Nec-5 (compound (1)) was also investigated. Compound (73), a 5-membered ring analog, retained most of the activity, while compound (74), a 7-membered ring analog, was less active. Compounds (77) and (78), having N-alkyl atoms incorporated within the 6-membered ring, were essentially inactive. This data indicates that increasing the size of the aliphatic ring of Nec-5 is inactivating, and is consistent with the side chain extension data discussed above. Interestingly, substitution of a phenyl ring for the cyclohexane ring (e.g., compound (76)) retained most of the activity.

2-methylthio-3-p-methoxyphenyl-5,6-disubstituted thieno[2,3-d]pyrimidin-4-ones (corresponding to compounds of Formula XIII) were prepared following the procedure of Scheme 2, using MeI instead of BrCH₂CN as the S-alkylation reagent, and are depicted in Table 6.

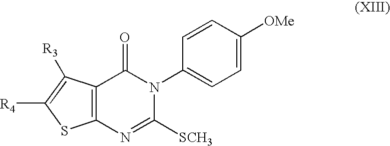

TABLE 6
Structure and activity of compounds of Formula (XIII)

| | | | Yield | | Max |
|---|---|---|---|---|---|
| Compound | R₃ | R₄ | (%) | EC₅₀ (μM) | Prot (%) |
| 79 | H | H | 86 | inactive | — |
| 80 | Me | H | 88 | inactive | — |
| 81 | Me | Me | 92 | inactive | — |
| 82 | Et | Me | 77 | inactive | — |
| 83 | Me | Et | 90 | inactive | — |
| 84 | Me | n-Pr | 92 | inactive | — |
| 85 | Me | i-Pr | 88 | inactive | — |
| 86 | Me | C₁₄H₂₉ | 81 | inactive | — |

| | | | | |
|---|---|---|---|---|
| 87 | —(CH₂)₃— | 92 | 0.24 | 97.0 |
| 88 | —(CH₂)₅— | 78 | inactive | — |
| 89 | —CH₂CH₂CHCH₃CH₂— | 74 | inactive | — |
| 90 | —CH═CHCH═CH— | 69 | 0.24 | 77 |
| 91 | —CH₂CH₂NEtCH₂— | 71 | inactive | — |
| 92 | —CH₂CH₂N(i-Pr)CH₂— | 66 | inactive | — |

As shown in Table 6, while the activities of the above compounds paralleled compounds of Formula XII, Table 5, the overall activities were generally lower. It is interesting to note that coordinated changes in the R₃, R₄, and R₁ groups resulted in surprising preservation of activity; for example, in the case of compound (87), the combination of a five-membered ring system (R₃ and R₄) and a methyl group (R₁) displayed higher activity than each change separately enacted (for example, compounds (6) and (73)), which may indicate somewhat different topology and orientation of Nec-5 analogs in the active site, and which depend on the combination of substituents in different parts of the molecule.

### Example 4

### Influence of Substituents on Sulfur and Thiophene Ring of Nec-5

As compound (73), bearing a 5-membered aliphatic ring in the R₃ and R₄ position, showed activity, synthesis of compounds of Formula (XIV) were prepared in order to determine if varying the R₁ group would translate into different SAR for the sulfur moiety. Reacting 2-mercapto-3-p-methoxyphenyl-5,6-trimethylenothieno[2,3-d]pyrimidin-4-one with RX in the presence of potassium hydroxide led to the formation of compounds of Formula (XIV), as depicted in Table 7.

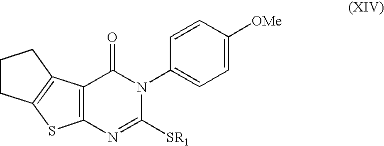

TABLE 7
Structure and activity of compounds of Formula (XIV)

| Compound | R₁ | Yield (%) | EC₅₀ (μM) | Max Prot (%) |
|---|---|---|---|---|
| 93 | Et | 88 | inactive | — |
| 94 | n-Pr | 87 | inactive | — |
| 95 | n-But | 91 | inactive | — |
| 96 | n-Pent | 78 | inactive | — |
| 97 | —CH₂CH═CH₂ | 76 | inactive | — |
| 98 | —CH₂C≡CH | 85 | 2.49 | 63.4 |
| 99 | —CH₂C₆H₅ | 84 | inactive | — |
| 100 | —CH₂(C₆H₄NO₂-4) | 76 | inactive | — |
| 101 | —CH₂COMe | 65 | inactive | — |
| 102 | —CH₂NO₂ | 71 | inactive | — |
| 103 | —CH₂CH₂OH | 77 | 7.14 | 100 |

As shown in Table 7, introduction of the five-membered ring generally maintained inactivity. Compounds (93) to (96), with an extended carbon chain, were inactive. Introduction of an electron withdrawing group (EWG), for example, as observed with compounds (100) to (102), completely eliminated activity.

The preparation of 2-mercapto 3-p-methoxyphenyl-5,6-disubstituted thieno[2,3-d]pyrimidin-4-ones (compounds of Formula (XV)) was carried out according to Scheme 2, with the exception that RX, in the presence of potassium hydroxide, was used as the alkylating agent.

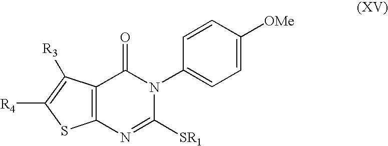

TABLE 8
Structure and activity of compounds of Formula (XV)

| | | | | Yield | EC₅₀ | MaxProt |
|---|---|---|---|---|---|---|
| Compound | R₁ | R₃ | R₄ | (%) | (μM) | (%) |
| 104 | —CH₂C≡CH | H | H | 85 | inactive | — |
| 105 | —CH₂C≡CH | Me | H | 77 | inactive | — |
| 106 | —CH₂C≡CH | Me | Me | 78 | 4.86 | 90.3 |
| 107 | —CH₂C≡CH | Me | Et | 79 | inactive | — |
| 108 | —CH₂C≡CH | Me | n-Pr | 81 | inactive | — |
| 109 | —CH₂C≡CH | —(CH₂)₅— | | 76 | inactive | — |
| 110 | Et | Me | Me | 93 | inactive | — |
| 111 | Et | Me | Et | 90 | inactive | — |
| 112 | Et | —(CH₂)₅— | | 90 | inactive | — |
| 113 | —CH₂CH₂CN | Me | Me | 49 | inactive | — |
| 114 | —CH₂CH₂OH | Me | Me | 55 | 7.26 | 78 |
| 115 | —CH₂CH₂OH | Me | Et | 80 | inactive | — |
| 116 | —CH₂CH₂OH | Me | n-Pr | 64 | inactive | — |
| 117 | Et | Me | n-Pr | 88 | inactive | — |
| 118 | —CH₂C(O)OH | Me | Me | 51 | inactive | — |

As shown in Table 8, compounds (106) and (114) possess some activity, but are significantly less potent than compound Nec-5. Notably, compound (110) was inactive, suggesting that the reduced size of the R₃ and R₄ substituents present on the thiophene ring did not translate into higher degree of flexibility in the tolerated size of the sulfur R₁ substituent.

### Example 5

### Influence of Substituents on Thiophene Ring and N-Pyrimidinone Part

Influence of the substituents of Nec-5 was studied by changing thiophene ring substituents R₃ and R₄ and the pyrimidinone part (R₁ and R₅) together. Since compounds (39), (48), (76), and (67), showed substantial activity, the synthesis of derivatives of these compounds was pursued.

3-p-Fluorophenyl-5,6-disubstituted thieno[2,3-d]pyrimidin-4-one-2-mercapto ethylcyanide compounds (corresponding to compounds of Formula (XVI) as depicted in Table 9) and the corresponding methylthioether compounds (corresponding to compounds of Formula (XVII), as depicted in Table 10) were generated, following a synthetic route similar to that of Scheme 3, upon reacting the corresponding thiol derivative with BrCH₂CN or MeI, respectively, in the presence of potassium hydroxide.

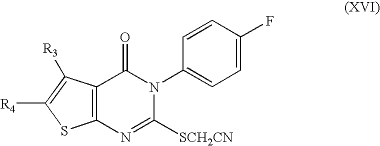

TABLE 9
Structure and activity of compounds of Formula (XVI)

| Compound | R₃ | R₄ | Yield(%) | EC₅₀(μM) | Max Prot(%) |
|---|---|---|---|---|---|
| 119 | Me | Me | 86 | 4.43 | 87.6 |
| 120 | Me | Et | 84 | inactive | — |

| | | | | |
|---|---|---|---|---|
| 121 | —(CH₂)₃— | 89 | 0.89 | 88.7 |
| 122 | —(CH₂)₅— | 88 | inactive | — |

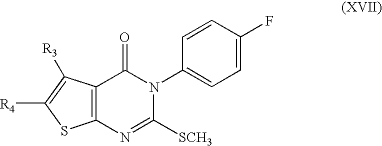

TABLE 10
Structure and activity of compounds of Formula (XVII)

| Compound | R₃ | R₄ | Yield(%) | EC₅₀(μM) | Max Prot(%) |
|---|---|---|---|---|---|
| 123 | Me | Me | 91 | 2.60 | 69.2 |
| 124 | Me | Et | 90 | inactive | — |

| | | | | |
|---|---|---|---|---|
| 125 | —(CH₂)₃— | 81 | 3.00 | 95 |
| 126 | —(CH₂)₅— | 74 | inactive | — |

As shown in Tables 9 and 10, seven-member ring containing molecules completely lacked activity (for example, compounds (122) and (126)), in contrast to the seven-member ring containing methoxy analog, compound (74). The latter result is reminiscent of the lack of activity displayed by compound (88). These results indicate that all three major moieties targeted by our analysis make important contributions to binding, and multiple unfavorable changes result in synergistic loss of activity, indicative of the inability of the resulting molecules to properly occupy the binding pocket.

Since 3-(3′,4′)-methylene-dioxyphenyl-5,6-tetramethylenothieno[2,3-d]pyrimidin-4-one-2-mercaptoethylcyanide (compound (48)), in which dioxolane ring is attached to the phenylmoiety, showed significant activity, the synthesis of its derivatives was carried out. 3-(3′,4′)-methylene-dioxyphenyl-5,6-disubstituted thieno[2,3-d]pyrimidin-4-one-2-mercapto ethylcyanide (corresponding to compounds of Formula (XVIII), as depicted in Table 11) and the corresponding methylthio ether (corresponding to compounds of Formula (XIX), depicted in Table 12) were generated by S-alkylation of the thiol derivative with BrCH₂CN or MeI in the presence of potassium hydroxide, respectively. 3-(3′,4′)-ethylene-dioxyphenyl-5,6-disubstituted thieno[2,3-d]pyrimidin-4-one-2′-mercapto ethylcyanide (corresponding to compounds of Formula (XX), as depicted in Table 13) were performed by the usual procedure of S-alkylation of corresponding thiol compounds.

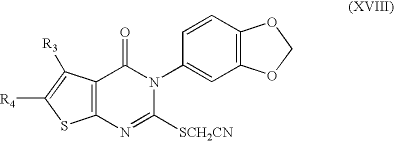

TABLE 11
Structure and activity of compounds of Formula (XVIII)

| Compound | R₃ | R₄ | Yield(%) | EC₅₀(μM) | Max Prot(%) |
|---|---|---|---|---|---|
| 127 | Me | Me | 91 | 1.65 | 100 |
| 128 | Et | Me | 89 | 1.90 | 100 |

| | | | | |
|---|---|---|---|---|
| 129 | —(CH₂)₃— | 86 | 1.18 | 100 |

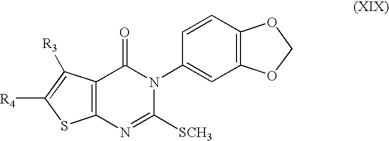

TABLE 12
Structure and activity of compounds of Formula (XIX)

| Compound | R₃ | R₄ | Yield(%) | EC₅₀(μM) | Max Prot(%) |
|---|---|---|---|---|---|
| 130 | Me | Me | 92 | 1.11 | 67.0 |
| 131 | Et | Me | 93 | inactive | — |

| | | | | |
|---|---|---|---|---|
| 132 | —(CH₂)₃— | 90 | inactive | — |

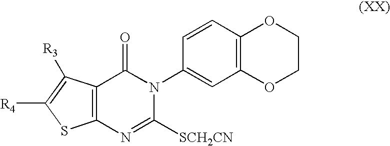

TABLE 13
Structure and activity of compounds of Formula (XX)

| | | | Yield | | Max |
|---|---|---|---|---|---|
| Compound | R₃ | R₄ | (%) | EC₅₀(μM) | Prot(%) |
| 133 | —(CH₂)₃— | | 91 | 0.25 | 100 |
| 134 | —(CH₂)₄— | | 90 | 0.22 | 100 |
| 135 | —(CH═CH—CH═CH)— | | 85 | 0.15 | 100 |
| 136 | Me | Me | 93 | 0.25 | 100 |

Experimental data in Tables 11, 12 and 13 shows that, with exception of inactive compounds (131) and (132), all of the compounds investigated, either with a methylene or ethylene dioxolane phenyl ring substituent, inhibited activity, and particularly for compounds of Formula (XX). In the latter case, consistent with our previous conclusion, there appears to be significant flexibility in the choice of R₅ phenyl ring substituents.

Compounds of Formula (XXI), depicted in Table 14, and compounds of Formula (XXII), depicted in Table 15, were synthesized in order to study the influence of the R₅ 3,4-dimethyl-benzene ring substituents on activity.

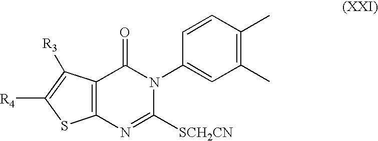

TABLE 14
Structure and activity of compounds of Formula (XXI)

| | | | | | Max |
|---|---|---|---|---|---|
| Compound | R₃ | R₄ | Yield(%) | EC₅₀(μM) | Prot(%) |
| 137 | Me | Me | 87 | 2.79 | 90.7 |

| | | | | |
|---|---|---|---|---|
| 138 | CH₂CH₂CH(CH₃)CH₂ | 81 | 16.7 | 38.0 |
| 139 | —(CH₂)₃— | 87 | inactive | — |

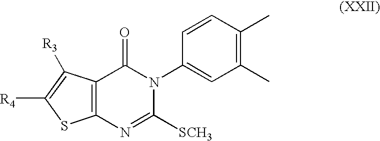

TABLE 15
Structure and activity of compounds of Formula (XXII)

| | | | | | Max |
|---|---|---|---|---|---|
| Compound | R₃ | R₄ | Yield(%) | EC₅₀(μM) | Prot(%) |
| 140 | Me | Me | 78 | inactive | — |

| | | | | |
|---|---|---|---|---|
| 141 | CH₂CH₂CH(CH₃)CH₂ | 85 | inactive | — |
| 142 | —(CH₂)₃— | 88 | inactive | — |

Interestingly, compound (137) showed higher activity than compound (43), providing the first example of coordinated changes to the left and right portion of the molecules displaying a compensatory, rather than a synergistic, effect. It is possible that a 3,4-Me-substituted molecule may assume an alternative binding position in the presence of the smaller R₃/R₄ substituents, resulting in this observed retention of the activity. However, this effect is limited to a particular combination of R₃/R₄, as compounds (138) and (139) are essentially inactive.

As compound (76) showed good activity, synthesis of its analogs with various phenyl ring substituents (compounds corresponding to Formulae (XXIII) and (XXIV), as depicted in Tables 16 and 17, respectively) was carried out, by reacting aryl isothiocyanate with NaOH in DMF to generate the corresponding thiol compound and, subsequently, S-alkylation with BrCH₂CN or MeI, respectively in the presence of potassium hydroxide to generate the desired target molecules.

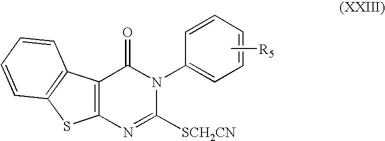

TABLE 16
Structure and activity of compounds of Formula (XXIII)

| Compound | R₅ | Yield(%) | EC₅₀(μM) | Max Prot(%) |
|---|---|---|---|---|
| 143 | H | 79 | ND^{a)} | ND^{a)} |
| 144 | 4-F | 77 | 3.06 | — |
| 145 | 4-OEt | 82 | inactive | — |
| 146 | 3,4-O₂(CH₂) | 82 | 0.27 | 100 |
| 147 | 4-OCF₃ | 83 | inactive | — |
| 148 | 4-NMe₂ | 67 | inactive | inactive |

^{a)}not determined

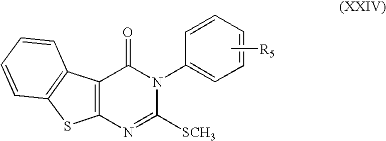

TABLE 17
Structure and activity of compounds of Formula (XXIV)

| Compound | R₅ | Yield(%) | EC₅₀(μM) | Max Prot(%) |
|---|---|---|---|---|
| 149 | H | 82 | ND^{a)} | ND^{a)} |
| 150 | 4-F | 78 | 3.06 | — |
| 151 | 4-OEt | 81 | inactive | — |
| 152 | 3,4-O₂(CH₂) | 88 | inactive | — |
| 153 | 4-OCF₃ | 76 | inactive | — |

^{a)}not determined

Compound (146) is a potent inhibitor, along with compounds (76) and (90), and this compound is consistent with previously defined SAR for other types of thiophene ring substituents. Therefore, substitution of phenyl for the cyclohexane ring does not appear to significantly change Nec-5 activity.

Since methyl groups in R₃ and R₄ positions showed significant activity (for example, compound (67)), analogs with additional phenyl ring substitutions (corresponding to compounds of the Formulae (XXV) and (XXVI), as depicted in Tables 18 and 19, respectively) were prepared by reacting corresponding the thiol derivative with BrCH₂CN or MeI, respectively, in the presence of potassium hydroxide.

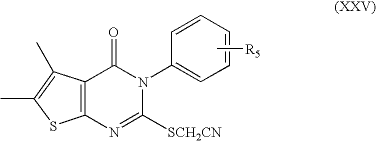

TABLE 18
Structure and activity of compounds of Formula (XXV)

| Compound | R₅ | Yield(%) | EC₅₀(μM) | Max Prot(%) |
|---|---|---|---|---|
| 154 | 4-OEt | 87 | 7.77 | 97 |
| 155 | 4-OBn | 79 | inactive | — |
| 156 | 4-OCF₃ | 81 | 3.70 | 63 |
| 157 | 4-NMe₂ | 83 | inactive | — |

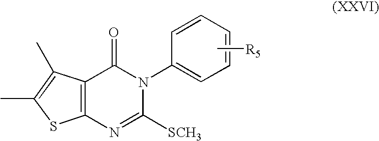

TABLE 19
Structure and activity of compounds of Formula (XXVI)

| Compound | R₅ | Yield(%) | EC₅₀(μM) | Max Prot(%) |
|---|---|---|---|---|
| 158 | 4-OEt | 90 | inactive | — |
| 159 | 4-OBn | 84 | inactive | — |
| 160 | 4-OCF₃ | 87 | inactive | — |
| 161 | 4-NMe₂ | 84 | inactive | — |

Analysis of compounds specified in Tables 18 and 19, as well as previously described derivatives with R¹=Me, R²=Me, suggest that such modifications are not favorable for activity, with all the analogs studied, exhibit generally less active than the corresponding cyclohexane moiety (R₃/R₄=—(CH₂)₄—) analogs of Nec-5. Furthermore, unlike results obtained with compounds of Formulae (XXI) and (XXII), synergistic loss of activity was observed for unfavorable changes in to the right part of the molecule and position R₄. For example, compounds (35) and (67) showed significant higher activity compared to compound (154).

For the study of the influence of the combination of the substituents on thiophene ring and N-pyrimidinone of Nec-5,3-aryl-5,6-disubstituted thieno[2,3-d]pyrimidin-4-one-2-mercapto ethylcyanide derivatives (corresponding to compounds of Formula (XXVII), as depicted in Table 20) were prepared.

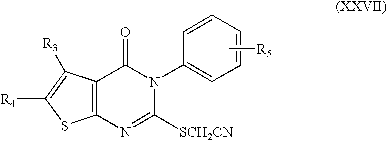

TABLE 20
Structure and activity of compounds of Formula (XXVII)

| Compound | R₃ | R₄ | R₅ | Yield(%) | EC₅₀(μM) | MaxProt(%) |
|---|---|---|---|---|---|---|
| 162 | H | H | H | 85 | inactive | — |

| | | | | | |
|---|---|---|---|---|---|
| 163 | —(CH₂)₃— | H | 90 | inactive | — |
| 164 | —(CH₂)₃— | 4-OEt | 67 | inactive | — |
| 165 | —(CH₂)₅— | H | 87 | inactive | — |
| 166 | —CH₂CH₂CH(CH₃)CH₂— | 3,4-(OMe)₂ | 78 | inactive | — |
| 167 | —(CH₂)₃— | 4-OCF₃ | 88 | 3.70 | 70 |
| 168 | —(CH₂)₅— | 4-OCF₃ | 85 | inactive | — |
| 169 | —(CH₂)₃— | 3,4-(OMe)₂ | 71 | inactive | — |

As shown in Table 20, combining changes to thiophene and phenyl rings was detrimental to activity, as only compound (167) showed some, albeit greatly reduced, activity.

### Example 6

### Influence of Substituents of Sulfur and N-Pyrimidinone of Nec-5

For the study of the influence of substituents on sulfur and N-pyrimidinone of Nec-5, 2-mercapto-3-aryl-5,6-tetramethylenothieno[2,3-d]pyrimidin-4-ones derivatives (corresponding to compounds of Formula (XXVIII), as depicted in Table 21), which combined unsubstituted phenyl ring and various substituents on sulfur, were prepared.

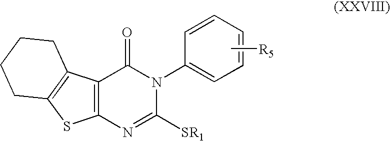

TABLE 21
Structure and activity of compounds of Formula (XXVIII)

| Compound | R₁ | R₅ | Yield(%) | EC₅₀(μM) |
|---|---|---|---|---|
| 170 | —CH₂CH═CH₂ | H | 58 | inactive |
| 171 | —CH₂Ph | H | 87 | inactive |
| 172 | —CH₂C₆H₄NO₂ | H | 83 | inactive |

As shown in Table 21, all the derivatives were found inactive, consistent with the requirement for the R₅ substituent to be para-methoxy, and with the preference for the R₁ substituent to be an ethylcyanide group.

### Example 7

### Influence of Substituents of Sulfur, N-Pyrimidinone as Well as Thiophene Ring

For the study of the influence of simultaneous substitution on sulfur in thiophene ring and N-pyrimidinone of Nec-5, 2-mercapto-3-aryl-5,6-disubstituted thieno[2,3-d]pyrimidin-4-one derivatives (corresponding to compounds of Formula (XXIX), as depicted in Table 22) were synthesized.

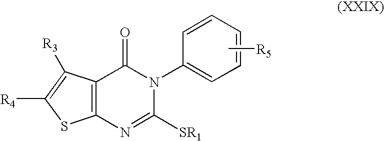

TABLE 22
Structure and activity of compounds of Formula (XXIX)

| Compound | R₁ | R₃ | R₄ | R₅ | Yield(%) | EC₅₀(μM) |
|---|---|---|---|---|---|---|
| 173 | Me | H | H | H | 90 | inactive |
| 174 | Me | CH₂CH₂CH(CH₃)CH₂ | | 3,4-(OMe)₂ | 88 | inactive |
| 175 | Et | —(CH₂)₅— | | H | 89 | inactive |
| 176 | CH₂C≡CH | Me | Me | 3,4-O₂(CH₂) | 69 | inactive |
| 177 | CH₂C≡CH | Et | Me | 3,4-O₂(CH₂) | 79 | inactive |
| 178 | Me | —(CH₂)₃— | | 4-OEt | 84 | inactive |
| 179 | Me | —(CH₂)₃— | | 3,4-O₂(CH₂) | 85 | inactive |
| 180 | CH₂CH₂OH | Et | Me | 3,4-O₂(CH₂) | 79 | inactive |

| | | | | | |
|---|---|---|---|---|---|
| 181 | Me | CH₂CH₂NEtCH₂ | H | 88 | inactive |
| 182 | Me | —(CH₂)₃— | 4-OCF₃ | 81 | inactive |

As shown in Table 22, consistent with the preference for R₃/R₄=—(CH₂)₄—, R₁=CH₂CN and R₅=para-OMe, all the analogs depicted in Table 22 were completely inactive.

### Analysis of Examples 1-7

Our preliminary SAR study demonstrated that the EC₅₀ value for inhibition of necroptosis in FADD-deficient Jacket T cells treated with TNFα of Nec-5 is closely related to the chemical structure of the molecule. The presence of the thioethylcyanide moiety (wherein R₁=ethylcyanide) at the α-position of the fused pyrimidone-4 ring is essential, as the replacement of this moiety for alternative groups results in complete loss of activity. An exception is when R₁ is methyl, as compound (6) exhibits the same EC₅₀ value as Nec-5, although it provides a significantly lower maximum protection value (of 71%). Oxidation of the sulfur atom, either to the sulfoxide (compound (30)) or to the sulfone (compound (31)) resulted in complete loss of activity. The presence of the R₅ para-methoxy group is also important, since variation of the electronic effect of the R₅ substituent, including modification of —OMe group, consistently gave compounds with lessened activity. Compounds with para-fluoro R₅ groups (for example, compound (39)) resulted in decreased activity, and a slightly decreased maximum protection (of 85.1%), while larger halides were not even tolerated. However, in the case of compound (135), an R₅ ethylene dioxy group is preferable to a para-methoxy, with compound (135) showing an almost 2-fold increase in activity. Finally, variation of the R₃/R₄ groups, for example, as observed with cyclopentyl compound (73), cycloheptyl compound (74), and even a R₃/R₄ benzene ring in compound (76), exhibited a certain degree of activity. It is worth pointing out that the introduction of two methyl groups to the R₃ and R₄ position significantly increases the activity. Additionally, these results suggest that there are positions in the molecule (for example, the R₅ position) that can be further studied in order to generate additional active Nec-5 analogs.

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A compound of Formula (I): 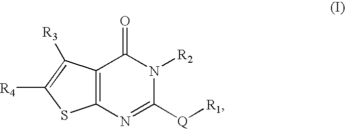
or a pharmaceutically acceptable salt or solvate thereof, wherein
Q is selected from the group consisting of —S—, —S(O)—, and —S(O)₂—;
R₁ is selected from the group consisting of C₁-C₉ alkyl, C₂-C₉ alkyenyl, C₂-C₉ alkynyl, C₆-C₁₂ aryl, and C₁-C₁₂ carbonyl;
R₂ is selected from the group consisting of C₁-C₉ alkaryl, and C₆-C₁₂ aryl; and
R₃ and R₄ are, independently, selected from the group consisting of C₁-C₉ alkyl, C₂-C₉ alkenyl, C₂-C₉ alkynyl, C₁-C₉ alkyloxy, and C₁-C₁₂ carbonyl, or R₃ and R₄, combined, form an C₃-C₉ carbocyclic, C₂-C₉ heterocyclic, C₆-C₁₂ aryl, or C₂-C₁₂ heteroaryl, ring system;
with the proviso that compounds wherein Q is —S—, R₁ is —CH₂CN, R₂ is —C₆H₄(4-OMe), and R₃ and R₄, combined, form an unsubstituted C₆-carbocyclic six-membered ring, are specifically excluded.

2. The compound of claim 1, said compound having the Formula (II): 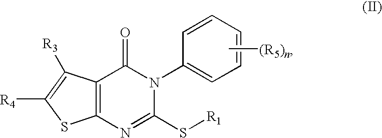
wherein
R₅ is selected from the group consisting of H, C₁-C₉ alkyl, C₂-C₉ alkenyl, C₂-C₉ alkynyl, C₃-C₉ carbocyclyl, C₂-C₉ heterocyclyl, C₂-C₉ heteroaryl, C₆-C₁₂ aryl, C₁-C₉ alkyloxy, C₁-C₉ alkylthio, C₆-C₁₂ arylthio, C₁-C₉ hydroxyalkyl, C₁-C₉ alkyloxy, C₆-C₁₂ aryloxy, C₁₋₁₂ carbonyl, C₁-C₉ fluoroalkyl, C₁-C₉ perfluoroalkyl, halogen, —SH, —OH, —N₃, —NH₂, —NO₂, and —CN; and n is 1, 2, 3, or 4;
or a pharmaceutically acceptable salt or solvate thereof.

3. The compound of claim 1, said compound having the Formula (III): 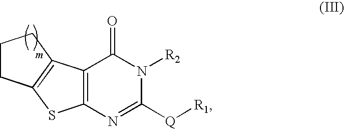
wherein
m is 1, 2 or 3;
or a pharmaceutically acceptable salt or solvate thereof.

4. The compound of claim 1, said compound having the Formula (IV): 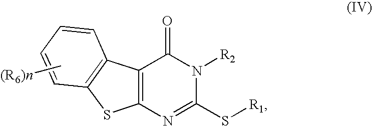
wherein
R₆ is selected from the group consisting of H, C₁-C₉ alkyl, C₂-C₉ alkenyl, C₂-C₉ alkynyl, C₃-C₉ carbocyclyl, C₂-C₉ heterocyclyl, C₂-C₉ heteroaryl, C₆-C₁₂ aryl, C₁-C₉ alkyloxy, C₁-C₉ alkylthio, C₆-C₁₂ arylthio, C₁-C₉ hydroxyalkyl, C₁-C₉ alkyloxy, C₆-C₁₂ aryloxy, C₁₋₁₂ carbonyl, C₁-C₉ fluoroalkyl, C₁-C₉ perfluoroalkyl, halogen, —SH, —OH, —N₃, —NH₂, —NO₂, and —CN; and n is 1, 2, 3, or 4;
or a pharmaceutically acceptable salt or solvate thereof.

5. The compound of claim 1, wherein said compound is selected from the group consisting of compounds 6 to 31 of Table 2; compounds 32 to 51 of Table 3; compounds 52 to 62 of Table 4; compounds 63 to 78 of Table 5; compounds 79 to 92 of Table 6; compounds 93 to 103 of Table 7; compounds 104 to 118 of Table 8; compounds 119 to 122 of Table 9; compounds 123 to 126 of Table 10; compounds 127 to 129 of Table 11; compounds 130 to 132 of Table 12; compounds 133 to 136 of Table 13; compounds 137 to 139 of Table 14; compounds 140 to 142 of Table 15; compounds 143 to 148 of Table 16; compounds 149 to 153 of Table 17; compounds to 154 to 157 of Table 18; compounds 158 to 161 of Table 19; compounds 162 to 169 of Table 20; compounds 170 to 172 of Table 21; and compounds 173 to 182 of Table 22;
or a pharmaceutically acceptable salt or solvate thereof.

6. The compound of claim 1, wherein said compound is selected from the group consisting of compounds 6, 13, 24, and 25 of Table 2; compounds 33 to 35, 38 to 41, 43, 44, and 47 to 49 of Table 3; compounds 53, 55, and 58 of Table 4; compounds 67, 68, and 72 to 76 of Table 5; compounds 87 and 90 of Table 6; compounds 98 and 103 of Table 7; compounds 106 and 114 of Table 8; compounds 119 and 121 of Table 9; compounds 123 and 125 of Table 10; compounds 127 to 129 of Table 11; compound 130 of Table 12; compounds 133 to 136 of Table 13; compounds 137 and 138 of Table 14; compounds 144 and 146 of Table 16; compound 150 of Table 17; compounds 154 and 156 of Table 18; and compound 167 of Table 20;
or a pharmaceutically acceptable salt or solvate thereof.

7. The compound of claim 6, wherein said compound is an active Nec-5 compound;
or a pharmaceutically acceptable salt or solvate thereof.

8. A pharmaceutical composition comprising a compound of Formula (I): 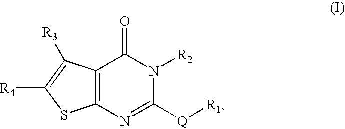
or a pharmaceutically acceptable salt or solvate thereof, wherein
Q is selected from the group consisting of —S—, —S(O)—, and —S(O)₂—;
R₁ is selected from the group consisting of C₁-C₉ alkyl, C₂-C₉ alkyenyl, C₂-C₉ alkynyl, C₆-C₁₂ aryl, and C₁-C₁₂ carbonyl;
R₂ is selected from the group consisting of C₁-C₉ alkaryl, and C₆-C₁₂ aryl; and
R₃ and R₄ are, independently, selected from the group consisting of C₁-C₉ alkyl, C₂-C₉ alkenyl, C₂-C₉ alkynyl, C₁-C₉ alkyloxy, and C₁-C₁₂ carbonyl, or R₃ and R₄, combined, form an C₃-C₉ carbocyclic, C₂-C₉ heterocyclic, C₆-C₁₂ aryl, or C₂-C₁₂ heteroaryl, ring system;
and a pharmaceutically acceptable excipient.

9. The pharmaceutical composition of claim 8, wherein said compound, or a pharmaceutically acceptable salt or solvate thereof, is selected from the group consisting of compounds 1 and 6 to 31 of Table 2; compounds 32 to 51 of Table 3; compounds 52 to 62 of Table 4; compounds 63 to 78 of Table 5; compounds 79 to 92 of Table 6; compounds 93 to 103 of Table 7; compounds 104 to 118 of Table 8; compounds 119 to 122 of Table 9; compounds 123 to 126 of Table 10; compounds 127 to 129 of Table 11; compounds 130 to 132 of Table 12; compounds 133 to 136 of Table 13; compounds 137 to 139 of Table 14; compounds 140 to 142 of Table 15; compounds 143 to 148 of Table 16; compounds 149 to 153 of Table 17; compounds to 154 to 157 of Table 18; compounds 158 to 161 of Table 19; compounds 162 to 169 of Table 20; compounds 170 to 172 of Table 21; and compounds 173 to 182 of Table 22.

10. The pharmaceutical composition of claim 9, wherein said compound, or a pharmaceutically acceptable salt or solvate thereof, is selected from the group consisting of compounds 1 and 6, 13, 24, and 25 of Table 2; compounds 33 to 35, 38 to 41, 43, 44, and 47 to 49 of Table 3; compounds 53, 55, and 58 of Table 4; compounds 67, 68, and 72 to 76 of Table 5; compounds 87 and 90 of Table 6; compounds 98 and 103 of Table 7; compounds 106 and 114 of Table 8; compounds 119 and 121 of Table 9; compounds 123 and 125 of Table 10; compounds 127 to 129 of Table 11; compound 130 of Table 12; compounds 133 to 136 of Table 13; compounds 137 and 138 of Table 14; compounds 144 and 146 of Table 16; compound 150 of Table 17; compounds 154 and 156 of Table 18; and compound 167 of Table 20.

11. The pharmaceutical composition of claim 10, wherein said compound, or a pharmaceutically acceptable salt or solvate thereof, is an active Nec-5 compound.

12. A method of synthesizing compounds of Formula (I-A): 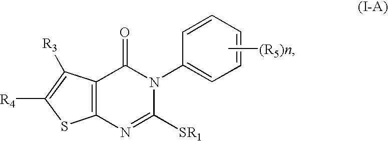
or a pharmaceutically acceptable salt or solvate thereof, wherein
R₁ is selected from the group consisting of H, C₁-C₉ alkyl, C₂-C₉ alkyenyl, C₂-C₉ alkynyl, C₆-C₁₂ aryl, and C₁-C₁₂ carbonyl;
R₂ is selected from the group consisting of C₁-C₉ alkaryl, and C₆-C₁₂ aryl; and
R₃ and R₄ are, independently, selected from the group consisting of C₁-C₉ alkyl, C₂-C₉ alkenyl, C₂-C₉ alkynyl, C₁-C₉ alkyloxy, and C₁-C₁₂ carbonyl, or R₃ and R₄, combined, form an C₃-C₉ carbocyclic, C₂-C₉ heterocyclic, C₆-C₁₂ aryl, or C₂-C₁₂ heteroaryl, ring system;
R₅ is selected from the group consisting of H, C₁-C₉ alkyl, C₂-C₉ alkenyl, C₂-C₉ alkynyl, C₃-C₉ carbocyclyl, C₂-C₉ heterocyclyl, C₂-C₉ heteroaryl, C₆-C₁₂ aryl, C₁-C₉ alkyloxy, C₁-C₉ alkylthio, C₆-C₁₂ arylthio, C₁-C₉ hydroxyalkyl, C₁-C₉ alkyloxy, C₆-C₁₂ aryloxy, C₁₋₁₂ carbonyl, C₁-C₉ fluoroalkyl, C₁-C₉ perfluoroalkyl, halogen, —SH, —OH, —N₃, —NH₂, —NO₂, and CN; and
n is 1, 2, 3, or 4;
wherein said method comprises providing a compound of Formula (I-B): 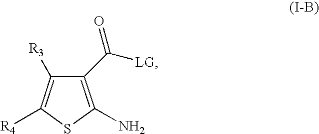
wherein
LG is C₁-C₉ alkyloxy, C₁-C₉ alkylsulfonyloxy, C₆-C₁₂ arylsulfonyloxy, or a halogen; and
reacting said compound of Formula (I-B) with an C₆-C₁₂ aryl isothiocyanate to provide a compound of Formula (I-C): 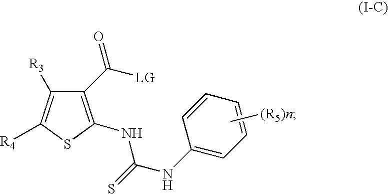
wherein said compound of Formula (I-C) is thereafter transformed to produce a compound of Formula (I-A).

13. The method of claim 12, wherein said compound of Formula (I-B) is obtained from a compound of Formula (I-E):

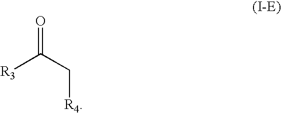

14. A method of treating a subject with a disease or condition, said disease or condition provided in Table 1, said method comprising administering to said subject an effective amount of a compound of claim 1, or a pharmaceutically acceptable salt or solvate thereof.

15. The method of claim 14, wherein said disease or condition is chronic, neurodegenerative disease; acute neurological disease; acute neurodegeneration; the result of cell death associated with renal failure; the result of retinal neuronal cell death; the result of cell death of cardiac muscle; the result of cell death of cells of the immune system; mycocardial infarction; cardiac infarction; stroke; ischemic stroke; hemorrhagic stroke; ischemia; ischemic liver disease, pancreatic disease, heart disease, brain disease, kidney disease or injury; ischemic mesenteric, retinal, or neuronal injury; ischemic injury during organ storage; delayed ischemic brain injury; traumatic brain injury; head trauma; sepsis; septic shock; necroptosis; necrosis; ischemic necrosis; retinal necrosis; necrotizing myopathy of intensive care; primary systemic infection; pancreatitis; or cell death induced by LPS.

16. The method of claim 15, wherein said chronic neurodegenerative disease is Alzheimer's disease; Huntington's disease; Parkinson's disease; amyotrophic lateral sclerosis; HIV-associated dementia; cerebral ischemia; amyotropic lateral sclerosis; multiple sclerosis; Lewy body disease; Menke's disease; Wilson's disease; Creutzfeldt-Jakob disease; or Fahr disease.

17. The method of claim 14, wherein said compound is selected from the group consisting of compounds 6 to 31 of Table 2; compounds 32 to 51 of Table 3; compounds 52 to 62 of Table 4; compounds 63 to 78 of Table 5; compounds 79 to 92 of Table 6; compounds 93 to 103 of Table 7; compounds 104 to 118 of Table 8; compounds 119 to 122 of Table 9; compounds 123 to 126 of Table 10; compounds 127 to 129 of Table 11; compounds 130 to 132 of Table 12; compounds 133 to 136 of Table 13; compounds 137 to 139 of Table 14; compounds 140 to 142 of Table 15; compounds 143 to 148 of Table 16; compounds 149 to 153 of Table 17; compounds to 154 to 157 of Table 18; compounds 158 to 161 of Table 19; compounds 162 to 169 of Table 20; compounds 170 to 172 of Table 21; and compounds 173 to 182 of Table 22;
or a pharmaceutically acceptable salt or solvate thereof.

18. The method of claim 17, wherein said compound is selected from the group consisting of compounds 6, 13, 24, and 25 of Table 2; compounds 33 to 35, 38 to 41, 43, 44, and 47 to 49 of Table 3; compounds 53, 55, and 58 of Table 4; compounds 67, 68, and 72 to 76 of Table 5; compounds 87 and 90 of Table 6; compounds 98 and 103 of Table 7; compounds 106 and 114 of Table 8; compounds 119 and 121 of Table 9; compounds 123 and 125 of Table 10; compounds 127 to 129 of Table 11; compound 130 of Table 12; compounds 133 to 136 of Table 13; compounds 137 and 138 of Table 14; compounds 144 and 146 of Table 16; compound 150 of Table 17; compounds 154 and 156 of Table 18; and compound 167 of Table 20;
or a pharmaceutically acceptable salt or solvate thereof.

19. The method of claim 18, wherein said compound is an active Nec-5 compound, or a pharmaceutically acceptable salt or solvate thereof.

20. A method of treating a subject with a disease or condition, said disease or condition provided in Table 1, said method comprising administering to said subject an effective amount of a pharmaceutical composition of claim 8.

21. The method of claim 20, wherein said disease or condition is chronic neurodegenerative disease; acute neurological disease; acute neurodegeneration; the result of cell death associated with renal failure; the result of retinal neuronal cell death; the result of cell death of cardiac muscle; the result of cell death of cells of the immune system; mycocardial infarction; cardiac infarction; stroke; ischemic stroke; hemorrhagic stroke; ischemia; ischemic liver disease, pancreatic disease, heart disease, brain disease, kidney disease or injury; ischemic mesenteric, retinal, or neuronal injury; ischemic injury during organ storage; delayed ischemic brain injury; traumatic brain injury; head trauma; sepsis; septic shock; necroptosis; necrosis; ischemic necrosis; retinal necrosis; necrotizing myopathy of intensive care; primary systemic infection; pancreatitis; or cell death induced by LPS.

22. The method of claim 21, wherein said chronic neurodegenerative disease is Alzheimer's disease; Huntington's disease; Parkinson's disease; amyotrophic lateral sclerosis; HIV-associated dementia; cerebral ischemia; amyotropic lateral sclerosis; multiple sclerosis; Lewy body disease; Menke's disease; Wilson's disease; Creutzfeldt-Jakob disease; or Fahr disease.

23. The method of claim 20, wherein said compound is selected from the group consisting of compound 1 and compounds 6 to 31 of Table 2; compounds 32 to 51 of Table 3; compounds 52 to 62 of Table 4; compounds 63 to 78 of Table 5; compounds 79 to 92 of Table 6; compounds 93 to 103 of Table 7; compounds 104 to 118 of Table 8; compounds 119 to 122 of Table 9; compounds 123 to 126 of Table 10; compounds 127 to 129 of Table 11; compounds 130 to 132 of Table 12; compounds 133 to 136 of Table 13; compounds 137 to 139 of Table 14; compounds 140 to 142 of Table 15; compounds 143 to 148 of Table 16; compounds 149 to 153 of Table 17; compounds to 154 to 157 of Table 18; compounds 158 to 161 of Table 19; compounds 162 to 169 of Table 20; compounds 170 to 172 of Table 21; and compounds 173 to 182 of Table 22;
or a pharmaceutically acceptable salt or solvate thereof.

24. The method of claim 23, wherein said compound is selected from the group consisting of compounds 1, 6, 13, 24, and 25 of Table 2; compounds 33 to 35, 38 to 41, 43, 44, and 47 to 49 of Table 3; compounds 53, 55, and 58 of Table 4; compounds 67, 68, and 72 to 76 of Table 5; compounds 87 and 90 of Table 6; compounds 98 and 103 of Table 7; compounds 106 and 114 of Table 8; compounds 119 and 121 of Table 9; compounds 123 and 125 of Table 10; compounds 127 to 129 of Table 11; compound 130 of Table 12; compounds 133 to 136 of Table 13; compounds 137 and 138 of Table 14; compounds 144 and 146 of Table 16; compound 150 of Table 17; compounds 154 and 156 of Table 18; and compound 167 of Table 20;
or a pharmaceutically acceptable salt or solvate thereof.

25. The method of claim 24, wherein said compound is an active Nec-5 compound;
or a pharmaceutically acceptable salt or solvate thereof.
